(19) United States
(12) Patent Application Publication
(10) Pub. No.: US 2026/0090764 A1
(43) Pub. Date: Apr. 2, 2026

(54) Tissue Repair System and Related Methods

(71) Applicant: Dunamis Medical Technologies, LLC, Greenville, AL (US)

(72) Inventor: PRITHVIRAJ CHAVAN, M.D., Greenville, AL (US)

(21) Appl. No.: 19/317,877

(22) Filed: Sep. 3, 2025

Related U.S. Application Data
(63) Continuation of application No. 18/023,367.
(60) Provisional application No. 63/071,105, filed on Aug. 27, 2020.

(51) Int. Cl. A61B 5/00; A61B 17/04

(52) U.S. Cl. CPC A61B 5/4848; A61B 5/4523; A61B 17/0401; A61B 2017/0404

(57) ABSTRACT

A knotless anchor assembly method and device comprising an anchor and a compression member, wherein the anchor further comprises a pilot tip and anchor body. During procedures to tension the knotless anchor assembly, the anchor is driven into bone or other surface or component, and the tension member is urged into the anchor body recess. The compression member is secured in the anchor body to lock the tension member in place. The knotless anchor assembly gives the surgeon or user the ability to optimize tensioning by having the ability to change tension in the tension member by adjusting the compression member during or after the repair.

### CROSS-REFERENCES TO RELATED APPLICATIONS

The present application is a continuation of U.S. patent application Ser. No. 18/023,367, filed Feb. 26, 2023, which is a national stage entry under 35 U.S.C. § 371 of PCT Application No. PCT/US21/48048, filed Aug. 27, 2021, which claims priority under 35 USC 119(e) to U.S. Provisional Patent Application Ser. No. 63/071,105, filed on Aug. 27, 2020, the entire contents of which are hereby incorporated by reference into this disclosure as if set forth fully herein.

### FIELD

The present invention is related to tissue repair. Specifically, the invention is related to an improved knotless anchor assembly and method of securing tissue, ligaments, sutures, grafts, allografts, membrane, gap fillers, tension members or bone to bone in stabilization procedures and real time tracking and monitoring of tissue repair and healing.

### BACKGROUND

Tendon, ligament and joint capsular injuries account for 45% of the orthopedic injuries which seek medical attention (Pennisi, 2002). Tendon injuries alone effect 30 million people annually (Wong, 2003), resulting in an enormous amount of physical and financial burden to both the individuals and the economy. Most of the affected people are treated with surgical repair or reconstruction to avoid long-term disability and pain relief. Tears can be primary and related to an injury or secondary and related to a re-tear after a primary repair or surgery. There has been an increased incidence of secondary tears and a large proportion of them may be avoided by using a better device to perform the procedure.

Secondary or type 2 failure occurs where the tendon fails medially to the repair, close to the muscle tendon attachment (Cho et al., 2011; Christoforetti et al., 2012; Kim et al., 2016;Kullar et al., 2015; Virk et al., 2017; Cho et al., 2010). Secondary medial cuff failure near the muscle tendon attachment after repair (type 2 failure) has been associated with the placement of knots and abrasive suture materials near the muscle tendon attachment, thus, potentially resulting in acute or chronic subacromial knot impingement, medial row stress concentration, tendon strangulation, and/or suture cut-out in this area (Cho et al., 2011; Christoforetti et al., 2012; Kim et al., 2016; Kullar et al., 2015; Virk et al., 2017). When failure occurs, the tendon/muscle is found to be torn medial to the previous repair site of the cuff tendon insertion (Cho et al., 2011;Christoforetti et al., 2012; Kim et al., 2016; Kullar et al., 2015; Virk et al., 2017).

Studies suggest that primary prevention can address secondary type 2 re-tears, and techniques such as utilizing knotless, linked and bridging constructs have been developed to prevent tissue strangulation and improve force distribution (Mazzocca et al., 2005; Denard et al., 2012). The use of tapes with these knotless repairs also decreases the compressive stress per unit area of tendon (Park et al., 2013; Vaishnav et al., 2010). The lack of knots avoids the possibility of acute or chronic postoperative knot impingement within the subacromial space (Yamakado et al., 2010), decreases operative time (Bukhart et al., 2009) and improves efficiency (Park, 2013; Park et al., 2014). Studies also suggest that rotator cuff tears that are repaired with a “tension overload mechanism” over a portion of the muscle-tendon units will undergo gradual failure with physiologic cyclic loading. Therefore, tendons should be repaired without tension if possible (Burkhart et al., 1997).

Several factors currently lead to an increased failure of repair. They are mainly a) knots causing impingement (Yamakado et al., 2010) and b) problems related to tensioning of repair, which can lead to strangulation of tissue causing necrosis over time, increased stresses on the anchoring device and failure with suture pulling out through tissue (Park, 2013; Park et al., 2014; Mazzocca et al., 2005; Denard et al., 2012).

Knotless anchors currently in the market only address knot related problems but continue to have other limitations whereby they either must be used in combination with another system, such as a secondary anchor, have limitation of the number of fibers passing through the anchor and continue to have major challenges related to tissue tensioning. These deficiencies hinder the surgeon's ability to adjust tissue tension at the time of repair or readjust once the repair is complete. This leads to under tensioning and failure due to gap formation or over-tensioning of the repair and failure at repair site, strangulation of blood supply to the tissue and re-tear. Thus, a need exists for a knotless anchor assembly that doesn't require a secondary anchor and allows for tension adjustment at the time of repair.

Soft tissue repair and other surgical procedures that require use of surgical components which are inserted in the body and remain there for a period of time to promote healing of an injury are well known in the art. However, it is difficult for medical professionals to track and monitor the progress of the healing of the soft tissue or other repaired member post-operation. If a soft tissue tears away from the main site or if a suture anchor is displaced after surgery, it may go unnoticed and the initial treatment plan adhered to, resulting in further and unnecessary damage to the tissue. For instance, some tears, such as rotator cuff tears, have a 20 to 30% re-tear rate. Thus, an efficient system and method for tracking and monitoring patient healing post-surgery is needed in the art.

### SUMMARY

Knotless assemblies that secure bone and tissue fragments can lose tension or over tension before the assemblies can lock or at the time of locking resulting in inefficient placement of the assembly during surgical procedures. The presently disclosed invention is a method and device comprising an anchor and a compression member, wherein the anchor further comprises a first end, second end and anchor body. During procedures to tension the knotless anchor assembly, the anchor is driven into bone, and the tension member is urged into the anchor body recess. The compression member is secured to the anchor body to lock the tension member in place. The knotless anchor assembly gives the surgeon or user the ability to optimize tensioning by having the ability to change tension in the tension member(s) by adjusting the compression member and does not require the use of an additional system, such as a secondary anchor.

A system and method for monitoring real-time healing of tissue and other members of the human body comprising one or more surgical devices or components, a monitoring component, and a detector is also described herein. The surgical components aid in the repair of injuries to tissue, tendons, or bone during surgical procedures remain in the body after insertion or implantation during a surgical procedure. Monitoring components incorporated in, in close proximity of, or affixed to the surgical components emit signals to detectors for tracking of the location and other information related to the surgical components, detailing location healing progress and allowing for real-time treatment and care.

In addition to the embodiments disclosed herein below, the present disclosure describes the following example embodiments.
Embodiment 1 is a system for monitoring healing of tissue or other members of a patient's body, comprising: (i) a surgical component utilized in a surgical procedure to aid in the repair of injuries to tissue, tendons, or bone, the surgical component configured to remain in the body for a period of time after completion of the surgical procedure; (ii) a monitoring component associated with the surgical component and having a traceable property that, when activated, is detectable to indicate a location of the monitoring component; and (iii) a detector configured to detect the activated traceable property of the activated monitoring component.
Embodiment 2 is the system of embodiment 1, wherein the surgical component comprises an anchor device.
Embodiment 3 is the system of embodiments 1 or 2, wherein the anchor device comprises at least one of a plate, metal polymer, bio-composite, suture, tape, textile, metal, fiber, and dye.
Embodiment 4 is the system of any of embodiments 1 through 3, wherein the monitoring component comprises at least one of a biosensor, scaffold, tissue, protein, amino acid, metal, and textile.
Embodiment 5 is the system of any of embodiments 1 through 4, wherein the traceable property comprises a biochemical reaction to one or more biochemical agents present in the proximity of the monitoring component.
Embodiment 6 is the system of any of embodiments 1 through 5, wherein the biochemical agent is at least one of injected by a user near the monitoring component to activate the traceable property; and produced by the patient's body during a healing process.

Embodiment 7 is the system of any of embodiments 1 through 6, wherein the traceable feature is an optically traceable feature of the monitoring component.

Embodiment 8 is the system of any of embodiments 1 through 7, wherein the monitoring component is one of attached to and integral with the surgical component.
Embodiment 9 is the system of any of embodiments 1 through 8, wherein the monitoring component is located on tissue near the surgical component.
Embodiment 10 is the system of any of embodiments 1 through 9, wherein the monitoring component contains a medicament configured to at least one of aid in the healing process, assist with pain management, and reduce inflammation near the surgical component.
Embodiment 11 is the system of any of embodiments 1 through 10, wherein the medicament is at least one of growth factors, steroids, antibiotics, and pain medication.
Embodiment 12 is the system of any of embodiments 1 through 11, wherein the monitoring component is configured to release the medicament in response to an activation signal received by the monitoring component.
Embodiment 13 is the system of any of embodiments 1 through 12, wherein the activation signal comprises a signal initiated by a user having an activation device.
Embodiment 14 is the system of any of embodiments 1 through 13, wherein the activation signal comprises a biochemical reaction with a biochemical agent present in the vicinity of the monitoring component.
Embodiment 15 is the system of any of embodiments 1 through 14, further comprising an electronic mobile device in communication with the detector, the electronic mobile device including a processor and a non-transient computer-readable storage media containing a set of instructions that, when executed by the processor, cause the electronic mobile device to: (i) receive data from the detector pertaining to the activated traceable component of the monitoring component; (ii) analyze the data pertaining to the activated traceable component of the monitoring component to determine the location of the activated monitoring component; and (iii) display, on a display device, the relative location of the activated monitoring component and the surgical component.
Embodiment 16 is the system of any of embodiments 1 through 15, wherein the set of instructions, when executed by the processor, further cause the electronic mobile device to determine the occurrence of at least one of a healing event and a re-tear event.
Embodiment 17 is the system of any of embodiments 1 through 16, wherein the detector forms a part of the electronic communication device.
Embodiment 18 is the system of any of embodiments 1 through 17, wherein the display device forms a part of the electronic communication device.
Embodiment 19 is the system of any of embodiments 1 through 18, wherein the electronic communication device comprises at least one of a smart phone, computer, laptop computer, and a tablet computer.
Embodiment 20 is a method of monitoring healing of tissue or other members in a patient's body, comprising the steps of (a) providing a system for monitoring healing of tissue or other members in the patient's body, the system comprising: (i) a surgical component configured for use in a surgical procedure to aid in the repair of injuries to tissue, tendons, or bone, the surgical component configured to remain in the body for a period of time after completion of the surgical procedure; (ii) a monitoring component associated with the surgical component and having a traceable property that, when activated, is detectable to indicate a location of the monitoring component; and (iii) a detector configured to detect the activated traceable property of the activated monitoring component; (b) implanting the surgical component into the patient's body during a surgical procedure to repair an injury to at least one of tissue, tendons, and bone; (c) implanting at least one of the monitoring components within tissue, tendons, or bone in the vicinity of the surgical component; (d) detecting the activated monitoring component; and (e) determining the location of the activated monitoring component; and (f) determining the occurrence of at least one of a healing event and a re-injury event based upon the determined location of the activated monitoring component.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1A is a partially exploded view of an example of a knotless anchor assembly, according to some embodiments;

FIG. 1B is a perspective view of the knotless anchor assembly of FIG. 1A, according to some embodiments;

FIG. 1C is a partially exploded view of an example of a compression member and anchor forming part of the knotless anchor assembly of FIG. 1A, according to some embodiments;

FIG. 2A is a perspective view of an example of a pilot tip forming part of the knotless anchor assembly of FIG. 1A, according to some embodiments;

FIG. 2B is a perspective view of another example of a pilot tip forming part of the knotless anchor assembly of FIG. 1A, according to some embodiments;

FIG. 3A is a partially exploded cross-sectional view of the compression member and anchor of FIG. 1C, according to some embodiments;

FIG. 3B is a perspective view of the knotless anchor assembly of FIG. 1A coupled with an example of an insertion sleeve, according to some embodiments;

FIG. 3C is a cross-sectional view of the knotless anchor assembly coupled with the insertion sleeve of FIG. 3B, according to some embodiments;

FIG. 3D is another perspective view of the knotless anchor assembly of FIG. 1A coupled with an example of an insertion sleeve, according to some embodiments;

FIG. 4A is a perspective view of the knotless anchor assembly of FIG. 1A coupled with an example of a broach/pusher, according to some embodiments;

FIG. 4B is another perspective view of the knotless anchor assembly of FIG. 1A coupled with an example of a broach/pusher, according to some embodiments;

FIG. 4C is a perspective view of the knotless anchor assembly of FIG. 1A in fully assembled configuration, according to some embodiments;

FIG. 5A is a cross-sectional view of the knotless anchor assembly of FIG. 1A coupled with an example of a passing loop, according to some embodiments;

FIG. 5B is a perspective view of the knotless anchor assembly of FIG. 1A coupled with an example of a passing loop, according to some embodiments;

FIG. 5C is another perspective view of the knotless anchor assembly of FIG. 1A coupled with an example of a passing loop, according to some embodiments;

FIG. 6A is a partially exploded plan view of another example of a knotless anchor assembly, according to some embodiments;

FIG. 6B is a cross-sectional view of the knotless anchor assembly of FIG. 6A, according to some embodiments;

FIG. 6C is a partially exploded perspective view of the knotless anchor assembly of FIG. 6A, according to some embodiments;

FIG. 6D is another partially exploded perspective view of the knotless anchor assembly of FIG. 6A, according to some embodiments;

FIG. 6E is a perspective view of the knotless anchor assembly of FIG. 6A in a fully assembled configuration, according to some embodiments;

FIG. 7A is a perspective view of another example of a knotless anchor assembly in a fully assembled configuration, according to some embodiments;

FIG. 7B is a partially exploded perspective view of the knotless anchor assembly of FIG. 7A, according to some embodiments;

FIG. 7C is a cross-sectional view of the knotless anchor assembly of FIG. 7A, according to some embodiments;

FIG. 7D is another cross-sectional view of the knotless anchor assembly of FIG. 7A, rotated 90° about the longitudinal axis from the view of FIG. 7C, according to some embodiments;

FIG. 7E is a cross-sectional view of an example of a proximal portion of a compression member forming part of the knotless anchor assembly of FIG. 7A, according to some embodiments;

FIG. 8A is a perspective view of an anchor forming part of the knotless anchor assembly of FIG. 7A, according to some embodiments;

FIG. 8B is a perspective view of the anchor of FIG. 8A coupled with an example of a passing loop and tension member, according to some embodiments;

FIG. 8C is another perspective view of the anchor of FIG. 8A coupled with an example of a passing loop and tension member, according to some embodiments;

FIG. 8D is a perspective view of the anchor of FIG. 8A coupled with a tension member, according to some embodiments;

FIG. 8E is a perspective of the knotless anchor assembly of FIG. 7A in a fully assembled configuration, according to some embodiments;

FIG. 9A is an exploded perspective view of the knotless anchor assembly of FIG. 7A, according to some embodiments;

FIG. 9B is a cross-sectional view of the knotless anchor assembly of FIG. 7A, according to some embodiments;

FIG. 9C is a plan view of the knotless anchor assembly of FIG. 7A, illustrating in particular the anchor implanted in bone during use, according to some embodiments;

FIG. 9D is another plan view of the knotless anchor assembly of FIG. 7A, illustrating in particular the fully assembled configuration implanted in bone during use, according to some embodiments;

FIG. 9E is a perspective view of the knotless anchor assembly of FIG. 7A coupled with an example of a guide post, according to some embodiments;

FIG. 10A is a perspective view of another example of a knotless anchor assembly in a fully assembled configuration, according to some embodiments;

FIG. 10B is a cross-sectional view of the knotless anchor assembly of FIG. 10A, according to some embodiments;

FIG. 10C is an exploded perspective view of the knotless anchor assembly of FIG. 10A coupled with a passing loop and a tension member, according to some embodiments;

FIG. 10D is an exploded perspective view of the knotless anchor assembly of FIG. 10A coupled with a tension member, according to some embodiments;

FIG. 10E is another perspective view of the knotless anchor assembly of FIG. 10A in a fully assembled configuration, according to some embodiments;

FIG. 11A is a partially exploded plan view of another example of a knotless anchor assembly, according to some embodiments;

FIG. 11B is a perspective view of the knotless anchor assembly of FIG. 11A in a fully assembled configuration, according to some embodiments;

FIG. 11C is a cross-sectional view of the knotless anchor assembly of FIG. 11A, according to some embodiments;

FIG. 11D is another cross-sectional view of the knotless anchor assembly of FIG. 11A, according to some embodiments;

FIG. 12A is a plan view of an example of an anchor forming part of the knotless anchor assembly of FIG. 1A, according to some embodiments;

FIG. 12B is another plan view of the anchor of FIG. 12A, according to some embodiments;

FIG. 13 is a perspective view of the anchor of FIG. 12A coupled with an example of an insertion tool, according to some embodiments;

FIG. 14 is a perspective view of the anchor of FIG. 12A coupled with another example of an insertion tool, according to some embodiments;

FIG. 15 is a perspective view of the anchor of FIG. 3A coupled with another example of an insertion tool, according to some embodiments;

FIG. 16A is a plan view of the anchor of FIG. 12A used with an example of a secondary compression member, according to some embodiments;

FIG. 16B is a plan view of the anchor of FIG. 12A used with another example of a secondary compression member, according to some embodiments;

FIG. 17 is a perspective view of the anchor of FIG. 3A assembled with a plurality of tension members, according to some embodiments;

FIG. 18 is a perspective view of a tension member forming part of the knotless anchor assembly of FIG. 1A coupled with a tissue and a passing loop, according to some embodiments;

FIG. 19A is a plan view of a tension member forming part of the knotless anchor assembly of FIG. 1A coupled with an example of an anchor member, according to some embodiments;

FIG. 19B is a side plan view of the anchor member of FIG. 19A, according to some embodiments;

FIG. 19C is a perspective view of the anchor member of FIG. 19A, according to some embodiments;

FIG. 19D is a plan view of the anchor member of FIG. 19A, rotated 90° from the view of FIG. 19B, according to some embodiments;

FIG. 20 is a schematic view of an example of a system of monitoring tissue health, according to some embodiments;

FIG. 21 is a block diagram of the system of monitoring tissue health of FIG. 20, according to some embodiments;

FIG. 22 is another schematic view the system of monitoring tissue health of FIG. 20, according to some embodiments; and

FIGS. 23-24 are block diagrams illustrating example computer systems with which any of the devices or systems described herein may be implemented, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The tissue repair system and related methods disclosed herein boasts a variety of inventive features and components that warrant patent protection, both individually and in combination.

Knotless anchors in the prior art hinder surgeons' ability to adjust tissue tension at the time of repair or readjust once the repair is complete resulting in under tensioning and failure due to gap formation or over-tensioning of the repair and failure at repair site, strangulation of blood supply to the tissue and re-tear. The knotless anchor assembly 10 disclosed herein allows the surgeon to adjust the repair tension based on the tissue biology of the tendon and, hence, optimizes the mechanical construct of the repair by providing a stable tension free repair. This reduces failures of repair due to inappropriate soft tissue tensioning. Further, surgical cost will be reduced due to decreasing the number of devices needed for the procedure, as the knotless anchor assembly 10 can be used independently and does not require other systems or anchors. The knotless anchor assembly 10 allows more sutures to pass through the anchor body and reduces the operative time since it eliminates the need for knot tying.

Additionally, the knotless anchor assembly can be used as a self-tapping anchor and can have multiple threaded components to increase the pull-out force. Several embodiments allow the surgeon or user performing the procedure to modify the repair during or after implanting the anchor body in the bone by adding more tension members and changing the size of the compression member, if required. Actively controlling the process of the repair during the repair provides an ultimate repair construct.

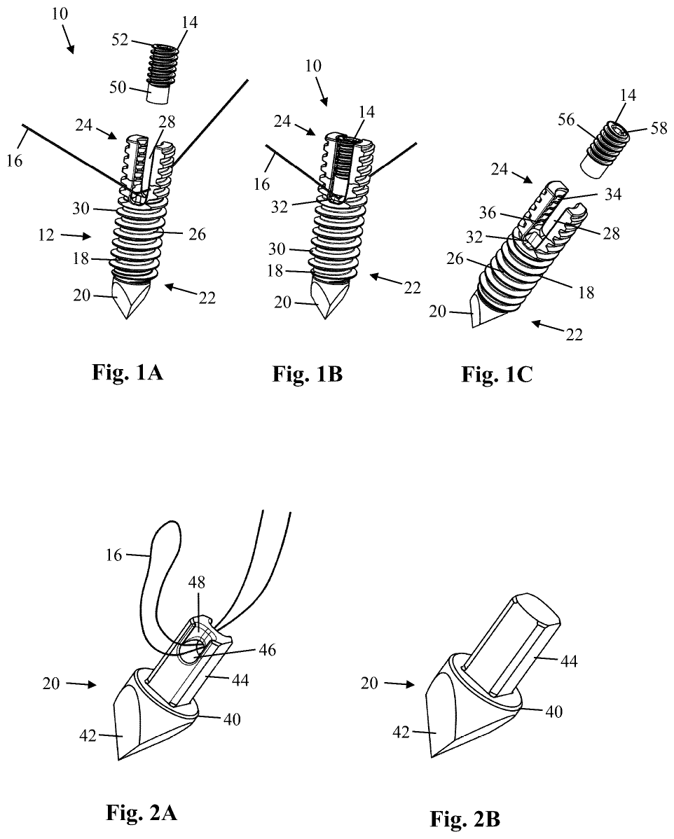

Referring to FIGS. 1A-3C, in some embodiments, the knotless anchor assembly 10 disclosed herein comprises an anchor 12, a compression member 14, and a tension member 16. By way of example only, the anchor 12 may be configured to securely penetrate a bony structure and lockingly receive at least a portion of the tension member 16 therein (which may be secured to the anchor 12 by way of the compression member 14), while at least another portion of the tension member 16 is secured to one or more of tissue, ligaments, sutures, grafts, allografts, membrane, gap fillers, tension members or bone, to thereby secure the one or more of tissue, ligaments, sutures, grafts, allografts, membrane, gap fillers, tension members or bone to the bony structure in a stabilization procedure.

In some embodiments, the anchor 12 may include an anchor body 18 defining a longitudinal axis and a pilot tip 20. By way of example only, the anchor body 18 further comprises a distal end 22, a proximal end 24, an outer surface 26, and an anchor body recess 28. In some embodiments, the distal end 22 includes a bone-engaging feature 30 positioned on or around the outer surface 26. By way of example only, the bone-engaging feature 30 may comprise at least one of a helical thread extending around the outer surface 26 (e.g. as shown in FIGS. 1A-3C), transverse or annular grooves or ridges, bumps, spikes, medical-grade adhesive, and/or a roughened surface. In some embodiments, the bone-engaging feature 30 may extend or be positioned around the cylindrical outer surface 26 for the entire length of the anchor body 18. In some embodiments, the bone-engaging feature 30 may extend or be positioned around the cylindrical outer surface 26 for less than the entire length of the anchor body 18.

In some embodiments, the anchor body 18 may include an anchor body recess 28 traversing at least part of the anchor body 18 between the proximal end 24 and distal end 22, and configured to receive at least a portion of the compression member 14 therein. In some embodiments, the anchor body recess 28 may comprise a longitudinal slot formed within the anchor body 18 from the proximal end 24 toward the distal end 22. In some embodiments, the anchor body recess 28 may have a base 32 and a cylindrical inner surface 34 including a mating thread 36 configured to receive the compression member 14. In some embodiments, the proximal end 24 of the anchor body 18 may be partial or complete, may have a cannulation slot or openings necessary for the knotless anchor assembly 10 to perform its function. Also, the base 32 of the anchor body recess 28 may be partial or complete or may have holes or slots. In some embodiments, the anchor body 18 may have a cannulation or inner lumen 38 extending distally from the base 32 of the anchor body recess 28 that is open to and in communication with the anchor body recess 28.

In some embodiments, the distal end 22 of the anchor 12 may further comprise a pilot tip 20 that leads the anchor 12 into the bone 30, or alternatively the anchor 12 can be implanted using a tool or be self-punching or otherwise self-engaging. In some embodiments, the pilot tip 20 may or may not assist with tensioning the knotless anchor assembly 10. In some embodiments, the pilot tip 20 may or may not be made out of the same material as the anchor 12 and can be made from other materials typically used in the industry. In some embodiments, the pilot tip 20 and anchor 12 may be manufactured as one unit using standard manufacturing process used in the industry, or the pilot tip 20 can be manufactured separately from the anchor 12 and later on assembled on the anchor body 18. In some embodiments, the pilot tip 20 may be different shapes depending on the force needed for insertion. In some embodiments, the pilot tip 20 may vary in length depending on the location and anatomy of the application.

In some embodiments, the pilot tip 20 may include a base 40 including a tapered leading surface 42 configured to penetrate into bone. The pilot tip 20 may further include a body 44 extending proximally from the base 40, the body 44 sized and configured for insertion within the cannulation or inner lumen 38 of the anchor body 18. In some embodiments, the body 44 may have a partial or complete transverse hole 46 formed therethrough, and a longitudinal recess 46 formed on one or more sides of the body 44, as shown in FIG. 2A. In such embodiments, the transverse hole 46 and/or longitudinal recess 48 are configured to enable a passing loop 60 or a portion of the tension member 18 to pass through the transverse hole 46 via the cannulation 38 of the anchor body 18. In some embodiments, the pilot tip 20 or part of the pilot tip 20 including the body 44 may be solid and non-cannulated, as shown in FIG. 2B. In some embodiments, the body 44 may be cannulated or have slots or grooves formed therein. In some embodiments, the pilot tip 20 may be manufactured in a process which requires it to be connected to the anchor 12, e.g. press fit, mold, twist, threads, overlay on the anchor 12 or by other processes of manufacturing to achieve the same outcome and function.

In some embodiments, the compression member 14 defines a longitudinal axis and further comprises a distal end 50 comprising a feature to couple the tension member 16 to the anchor body 18, and a proximal end 52 comprising a feature to couple to an insertion instrument (e.g. driver). In some embodiments, the compression member 14 is generally cylindrical and has a size that is complementary to the anchor body recess 28 so that the compression member 14 may be securely mated to the anchor body 18 (e.g., by coupling within the anchor body recess 28). In some embodiments, the distal end 50 comprises a cylindrical base 54 having a smooth surface configured to contact the tension member 16 and further configured to create pinch points between the cylindrical base 54 and the base 32 of the anchor body recess 28 (for example) to secure the tension member 16 to the anchor 12. In some embodiments, the proximal end 52 of the compression member includes a mating thread 56 configured to mate with the mating thread 36 of the anchor body recess 28 to securely mate the compression member 14 and the anchor 12. In some embodiments, the proximal end 52 includes a proximal recess 58 configured to engage with an insertion instrument. In some embodiments, the compression member 14 may be comprised of multiple individual components.

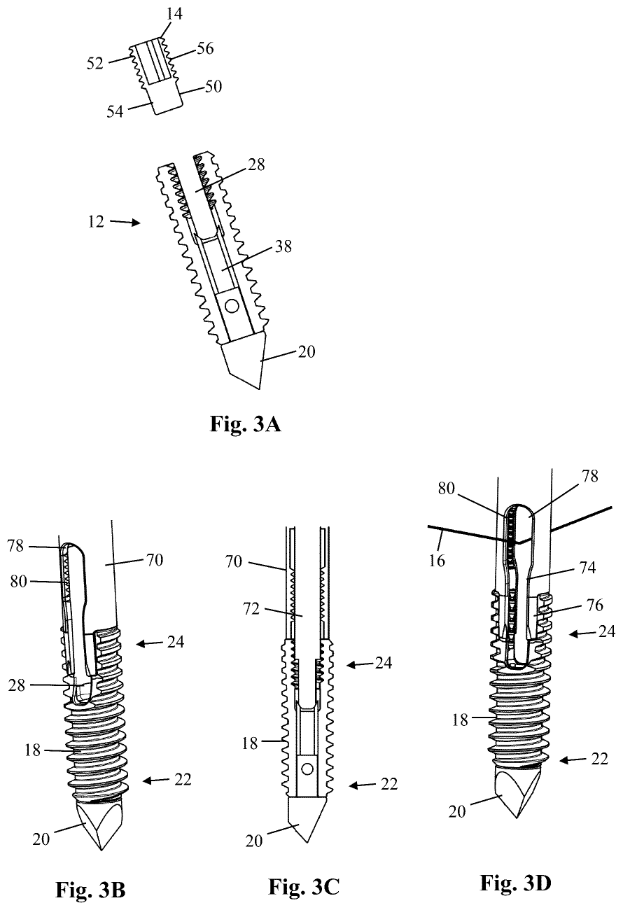

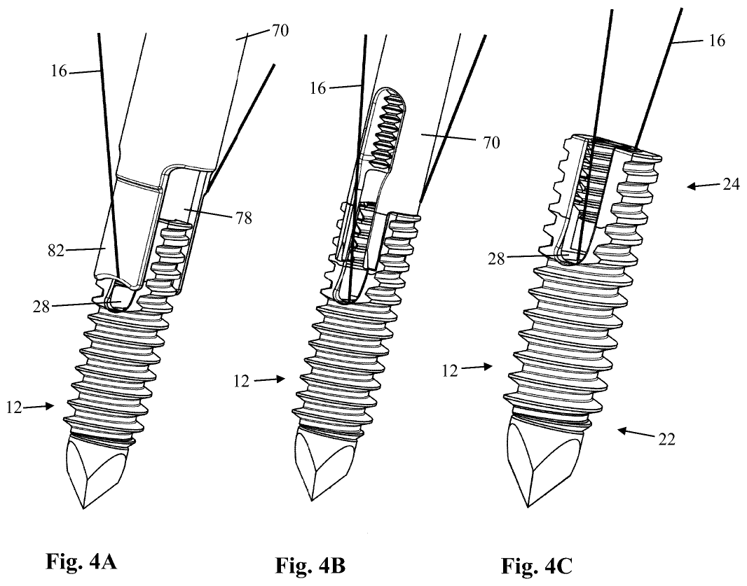

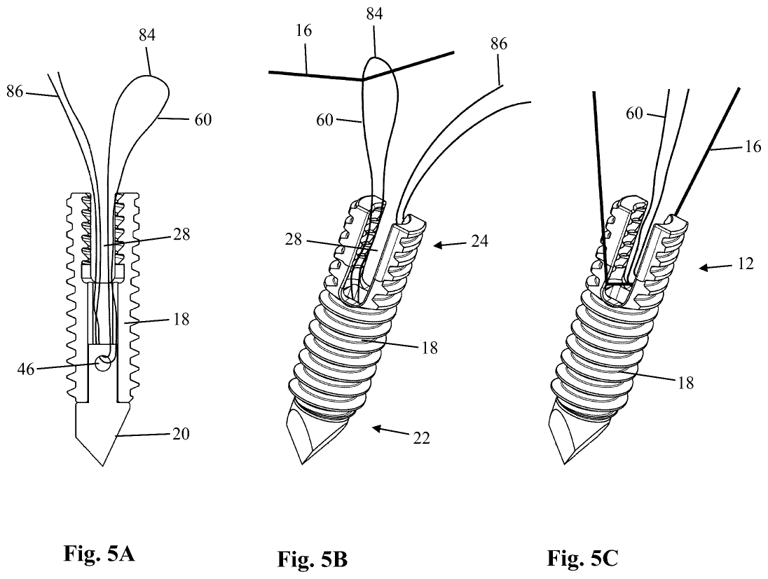

In some embodiments, the tension member 16 may pass through the anchor body recess 28 directly or pass through the anchor body recess 28 and the transverse hole 46 in the body 44 of the pilot tip 20, as shown in FIGS. 2A and 5A. In some embodiments, the tension member 16 can be part of suture material or fixation member of soft tissue. In some embodiments, a passing loop 60 (e.g. which may be used to facilitate coupling of the tension member 16 and the anchor 12) may pass through the anchor body recess 28 directly or pass through the anchor body recess 28 and the transverse hole 46 in the body 44 of the pilot tip 20, as shown in FIGS. 5A and 5B. In some embodiments, subsequent pulling of the passing loop 20 urges the tension member 16 into the anchor body recess 28 and/or the transverse hole 46 in the body 44 of the pilot tip 20. In some embodiments, the compression member 14 may be a part of the knotless anchor assembly 10 at the time of anchor 12 implantation, as shown FIG. 1B, or it can be part of the tool or insertion sleeve 70 shown in FIGS. 3 and 4 or similar tool that be used as a single tool connected to the anchor 12 or multiple tools that can connect to the anchor body 18. After deployment, the compression member 14 may finally end up in the anchor body 18 to form a knotless anchor assembly 10.

By way of example only, to utilize the knotless anchor assembly 10, the anchor 12 may be driven into bone 5 lead by the pilot tip 20, and the tension member 16 may be urged into the anchor body recess 28 (e.g. with or without use of a passing loop 60). The compression member 14 may then be advanced into anchor body recess 28, for example such that the mating thread 56 of the compression member 14 mates with the mating thread 36 of the anchor body 18 (or via an alternative mating arrangement) to lock the tension member 16 in place, as shown in FIGS. 1B and 5C. If the user is dissatisfied with the tension in the tension member 16, then backing out the compression member 14 at least partially or completely unlocks the tension member 16, allowing for readjustment before re-locking. In some embodiments, the anchor 12 can be used with a different assembly tool used to implant the anchor 12 or knotless anchor assembly 10. In some embodiments, other techniques besides a coupling instrument or tool may be used to drive the compression member 14 to cause locking of the knotless anchor assembly 10.

For the first embodiment of the knotless anchor assembly 20 depicted in FIG. 1, the compression member 14 may secure the tension member 16 or other fixation member that passes through the anchor body recess 28 by downward migration using multiple mechanisms, including, but not limited to, threading, to secure the tension member(s) 16 using the compression member 14. The locking or securing of the tension member 16 may happen in multiple locations. For example, the knotless anchor assembly 10 may include pinch points between the compression member 14 and the anchor body 18 (e.g., between the cylindrical base 54 of the compression member 14 and the base 32 of the anchor body recess 28 as described above) or a combination of pinch points and compression by the distal end 50 of the compression member 14 or distal end 50 of the compression member 14 sitting on a base 32 of the anchor body recess 28, or it can be a combination of fixation between pinch points as described herein and the proximal end 38 of the anchor body 18 or a base within the anchor body recess 28 or other fixation point that may be created by this downward migration of the compression member 14. The location of the fixation points or base within the anchor body recess 28 or location of security of the tension members may vary within the anchor 12 itself.

In some embodiments, the compression member 14 may be inserted later or replaced with one of a different size, using a separate tool for its insertion. In such an embodiment, the compression member 14 is not part of the anchor body 18, insertion sleeve 70 or any other tool utilized to operate the knotless anchor assembly 10. In some embodiments, the compression member may be designed to lock the tension member 16 shown in FIG. 1B, tension member 16 and/or passing loop 60 depicted in FIG. 5C or any other member used as a fixation member.

In some embodiments, additional instruments can be utilized in this method by attaching an insertion sleeve 70 to the anchor body 18 and inserting a driver 72 through the insertion sleeve 70 and into the anchor body recess 28, as shown in FIGS. 3A-3D. In this embodiment, the driver 72 and insertion sleeve 70 assembly are used to introduce the anchor body 18 in the bone 5 or similar tissue that would act as a fixation point until the anchor body 18 is completely below the surface of the bone 5 or similar tissue. The driver 72 may then be removed while leaving the insertion sleeve 70 attached to the anchor body 18. With the insertion sleeve 70 attached to the anchor body 18 in this fashion, the tension member 16 may be inserted into the sleeve slot 74.

In some embodiments, the insertion sleeve 70 may have a mating component configured to mate with a corresponding mating component on the anchor body 18. For example, in some embodiments, the insertion sleeve 70 may have a threaded outer surface 76 configured to mate with the threaded inner surface 34 of the anchor body recess 28.

In some embodiments, the anchor 12 may be introduced in the bone 5 or tissue with the driver 72 or insertion sleeve 70 only.

In some embodiments, the tension member 16, passing loop 60 or compression member 14 may be attached or connected to the anchor body 18 or knotless suture anchor assembly 10 at the time of implantation of the anchor 12. In such embodiments, the compression member 14 may or may not be connected to the introducing device.

The insertion sleeve 70 may function as a driver or inserter to insert the anchor 12 into the bone 5 or any other anatomy that requires a fixation device to perform a repair. The length of the insertion sleeve 70 may vary depending upon the anatomical location, technique, tools used, type of surgery or other indications that may determine the length of the insertion sleeve 70. The insertion sleeve 70 may be manufactured using materials used to manufacture the knotless anchor assembly 10 that are defined herein but not limited to those materials and made by any manufacturing process used to manufacture anchor, tools or other typically used in the industry.

The amount of contact between the insertion sleeve 70 and the anchor body 18 may vary depending on the amount of strength and contact area necessary for optimal insertion of the knotless anchor assembly 10. In some embodiments, the insertion sleeve 70 may be partially or completely cannulated or in instances may be solid with no cannulation. The size of sleeve recess 78 may vary depending on the fibers that may pass through the insertion sleeve 70. The length of the sleeve recess 78 may vary and it may be partial or completely open on one or both sides or many surfaces. In some embodiments, the inner surface 80 of the insertion sleeve 70 may be partially or completely threaded or in the instances where the sleeve is solid with no cannulation the insertion sleeve 70 may not be threaded. In those instances, the insertion sleeve 70 may also function as a driver to insert the anchor 12.

Similarly, the dimensions of the anchor body recess 28 may vary depending on a variety of factors, including but not limited to the anatomy, location of repair its designed for, the number of fibers that may be needed to perform the repair, the surface area needed (e.g., the amount of surface area needed for sleeve contact), the strength of device depending on biomaterial or other anchor manufacturing material used, and the amount of contact if needed with the compression member 14. In some embodiments, the anchor body recess 28 may assist with the passage of fixation members or tissue to complete the repair.

The lengths and dimensions of the driver 72 may vary depending on the anatomical locations, techniques, tools or other factors that may influence the size of the driver 72. In some embodiments, the driver 72 may be solid with no cannulation or may be cannulated partially or completely or may have thread internally or externally. In some embodiments, the external surface area may be modified depending on the size of the knotless anchor assembly 10 and the anatomical location its used in to improve the safety of the device and prevent failures while performing the repair.

As shown in FIGS. 4A-4C, in some embodiments a broach/pusher 82 may be used to broach a passage into bone 5 while also urging the tension member 16 into the anchor body recess 28 of the implanted anchor 12. In some embodiments, the compression member 14 may be inserted into the insertion sleeve 70 and then threaded into the anchor 12. As described above, tightening or advancing the compression member 14 results in locking the tension member 16 inside the anchor body 18, while reversing or backing up the compression member 14 unlocks the tension member 16 to allow for adjusting its position (i.e. re-tensioning) before relocking by again advancing the compression member 14 distally. The insertion sleeve 70 may then be removed. In some embodiments, the compression member 14 may be deployed or engaged with the tension member 16 with any secure mechanism other than threading.

In some embodiments, the compression member 14 may be connected to the anchor body 18 or may not need a secondary introduction process.

In some embodiments, the anchor body 18 or knotless anchor assembly 10 may be driven in the bone 5 or a surface needing fixation without an internal driver 72.

In some embodiments, the broach/pusher 82 can be made out of the similar material used to manufacture the knotless anchor assembly 10 disclosed herein, but not limited to those materials, and may be made of other devices used in the industry.

In some embodiments, the broach/pusher 82 may be used to create channel in the soft tissue, bone, membrane, or graft. In some embodiments, the broach/pusher 82 may also be used to transport the tension member 16 for further locking by the compression member 14. In some embodiments, the broach/pusher 82 may also help with aligning of fibers or tension member 16 for optimal locking. In some embodiments, the broach/pusher 82 may also be a part of the tool construct used to insert the anchor 12. In some embodiments, the broach/pusher 82 may be used to create the channel or transport sutures or any other defined functions using different techniques which may vary from tapping, twisting but not limited to them. In some embodiments, the broach/pusher 82 may be used independently of other tools e.g. absence of insertion sleeve 70 to assist with the defined functions of the broach/pusher 82. By way of example only, the length, size, dimensions, design features may vary depending upon the anatomical location as long as the broach/pusher 82 is able to perform the defined functions. In some embodiments, the broach/pusher 82 may be manufactured as one piece or two pieces or a multiple piece construct. In some embodiments, the broach/pusher 82 may be partially or completely cannulated.

In some embodiments, the tension member 18 may be guided into the anchor 12 (e.g. the anchor body recess 28 and/or the transverse hole 46 of the pilot tip 20) using a passing loop 60, as shown by way of example only in FIGS. 5A-5C. For example, the anchor 12 may driven into the bone 5 using an insertion sleeve 70 and/or driver 72 as described above. The tension member 16 may be inserted into the looped end 84 of the passing loop 60, as shown in FIG. 5B. Next, the user may pull on the free ends 86 of the passing loop 60 to direct the tension member 16 into the anchor body recess 28, as shown in FIG. 5C. The compression member 14 may then be threaded into the anchor body 18 to lock the tension member 16 in place. In some embodiments, the passing loop 60 may be pulled out of the anchor 12 or left in place.

In some embodiments, the compression member 14 may unlock the tension member 16 or fixation members by backing up or similar mechanism to disengage or loosen the tension in the fixation/tension members 16. In some embodiments, the passing loop 60 may have a looped end 84 and free ends 86 or may have loop on both ends or can be any mechanism of transporting the fixation or tension member or members 16 in the anchor body recess 28 or within the anchor body 18 for locking purposing by the compression member 14.

By way of example only, FIG. 5A depicts the passing loop 60, passing through the transverse hole 46 on the pilot tip 20. In some embodiments, the passing loop 60 or member designed for similar mechanism may be connected to the anchor body 18 or be with or in close proximity and enable passing of the tension members 16 for locking using the compression member 14. As previously described, reversing or backing up the compression member 14 allows unlocking of the tension member 16 and adjustment of its position (i.e. re-tensioning) before relocking it in place. The locking between the anchor body 18 and the compression member 14 may enable fixation of the tension members 16, fixation members, bone 5, graft, soft tissue, and/or gap fillers. In some embodiments, the compression member 14 may interact with any of the aforementioned members to assist with fixation and repair/reconstruction for all embodiments taught herein.

In some embodiments, it is not necessary for the tension members 16 or the passing loop 60 to pass through the transverse hole 46 in the pilot tip 20 to perform its function which mainly is to position the tension members 16 in the anchor body recess 28 for locking using the compression member 14.

In some embodiments, the passing loop 60 may have a looped end 84 and at least one free end 86. In some embodiments, the passing loop 60 may have single or multiple loops. In some embodiments, the passing loop 60 may or may not have free ends. In some embodiments, the passing loop 60 may be used to pass the tensioning member, graft, allografts, membrane, and/or gap fillers. In some embodiments, the passing loop 60 at times may be a part of the repair when locked with the compression member 14.

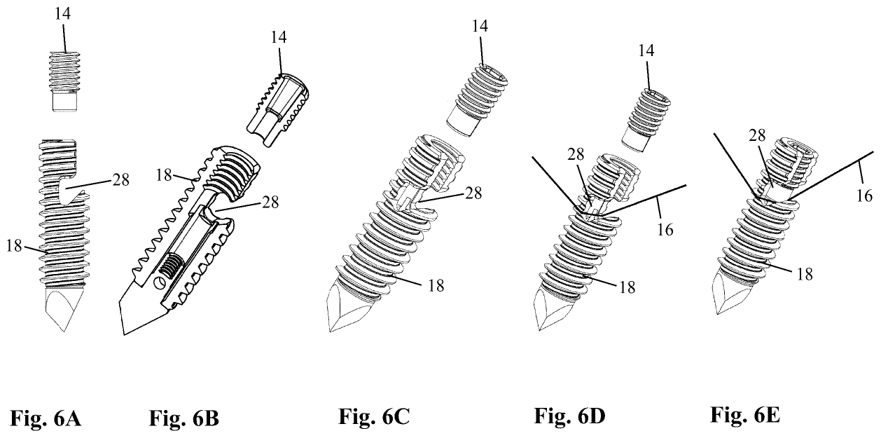

In some embodiments, the anchor body recess 28 may be partially off-center and have a generally J-shape, as shown by way of example only in FIGS. 6A-6E. In some embodiments, the tension member 16 may be passed into the anchor body recess 28 from the side of the anchor body 18, as shown by way of example in FIG. 6D.

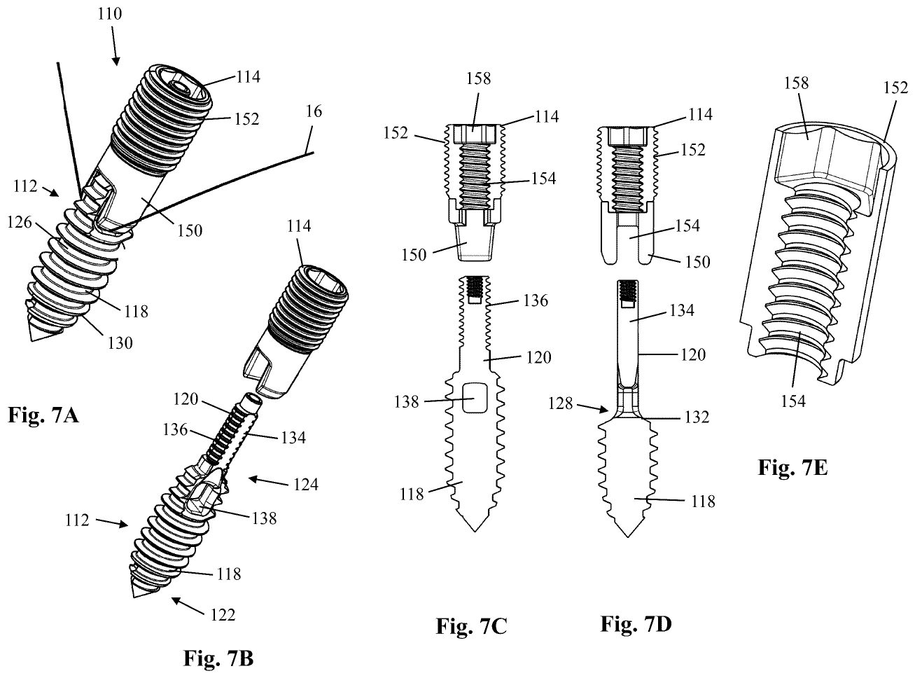

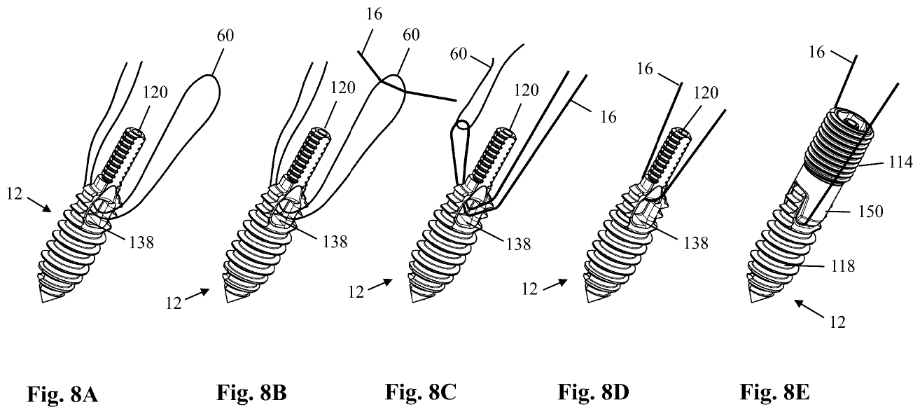

Referring to FIGS. 7A-8E, in some embodiments, the knotless anchor assembly 110 disclosed herein comprises an anchor 112, a compression member 114, and a tension member 16. By way of example only, the anchor 112 may be configured to securely penetrate a bony structure and lockingly receive at least a portion of the tension member 16 therein (which may be secured to the anchor 112 by way of the compression member 114), while at least another portion of the tension member 16 is secured to one or more of tissue, ligaments, sutures, grafts, allografts, membrane, gap fillers, tension members or bone, to thereby secure the one or more of tissue, ligaments, sutures, grafts, allografts, membrane, gap fillers, tension members or bone to the bony structure in a stabilization procedure.

In some embodiments, the anchor 112 may include an anchor body 118 defining a longitudinal axis. By way of example only, the anchor body 118 further comprises a distal end 122, a proximal end 124, an outer surface 126, and an anchor body recess 128. In some embodiments, the distal end 122 includes a bone-engaging feature 130 positioned on or around the outer surface 126. By way of example only, the bone-engaging feature 130 may comprise at least one of a helical thread extending around the outer surface 126 (e.g. as shown in FIGS. 7A-7B), transverse or annular grooves or ridges, bumps, spikes, medical-grade adhesive, and/or a roughened surface. In some embodiments, the bone-engaging feature 130 may extend or be positioned around the cylindrical outer surface 126 for the entire length of the anchor body 118. In some embodiments, the bone-engaging feature 130 may extend or be positioned around the cylindrical outer surface 126 for less than the entire length of the anchor body 118. In some embodiments, the distal end 122 of the anchor 112 may further comprise a tip that leads the anchor 112 into the bone 5, or alternatively the anchor 112 can be implanted using a tool or be self-punching or otherwise self-engaging.

In some embodiments, the proximal end 124 of the anchor body 118 comprises a post 120 extending proximally from the distal end 122 along the longitudinal axis, the post 120 configured to translatably receive the compression member 114. In some embodiments, the post 120 may include have a pair of planar outer surfaces 134 on opposite sides of the post 120, and a pair of threaded outer surfaces including mating threads 136 on opposite sides of the post 120, interspersed with the planar outer surfaces 134. In some embodiments, the anchor body 118 may include an anchor body recess 128 traversing at least part of the anchor body 118 between the proximal end 124 and distal end 122, and configured to receive at least a portion of the compression member 114 therein. In some embodiments, the anchor body recess 128 may comprise a longitudinal cutaway around the post 120. In some embodiments, the anchor body recess 128 may have a base 132. In some embodiments, the proximal end 124 of the anchor body 118 may be partial or complete, may have a cannulation slot or openings necessary for the knotless anchor assembly 110 to perform its function. Also, the base 132 of the anchor body recess 128 may be partial or complete or may have holes or slots. In some embodiments, the anchor body 118 may have a transverse hole 138 formed therein for passage of the tension member 16 therethrough.

In some embodiments, the compression member 114 defines a longitudinal axis and further comprises a distal portion 150 comprising a feature to couple the tension member 16 to the anchor body 118, a proximal portion 152 comprising a feature to couple to an insertion instrument (e.g. driver), and in inner longitudinal channel 154 configured to receive the post 120 therein, as shown in FIGS. 7A-7E. In some embodiments, the distal portion 150 can rotate relative to the proximal portion 152 but is prevented from rotating when the compression member 114 is engaged with the post 150, as shown by way of example only in FIGS. 7A-8E. In some embodiments, the proximal portion 152 has mating threads configured to mate with the threaded outer surface 136 of the post 120. For example, when the compression member 114 is attached to the post 120 of the anchor body 118, the proximal portion 152 threads onto post 120, while the distal portion 150 engages the smooth outer surface 134 of the post 120 and translates along the post 120 linearly without rotating, as shown in FIGS. 7A, 7D and 8E. In some embodiments, the proximal portion 152 has a threaded outer surface for gaining purchase within bone. In some embodiments, the proximal portion 152 may have a smooth outer surface, as shown by way of example in FIG. 11D.

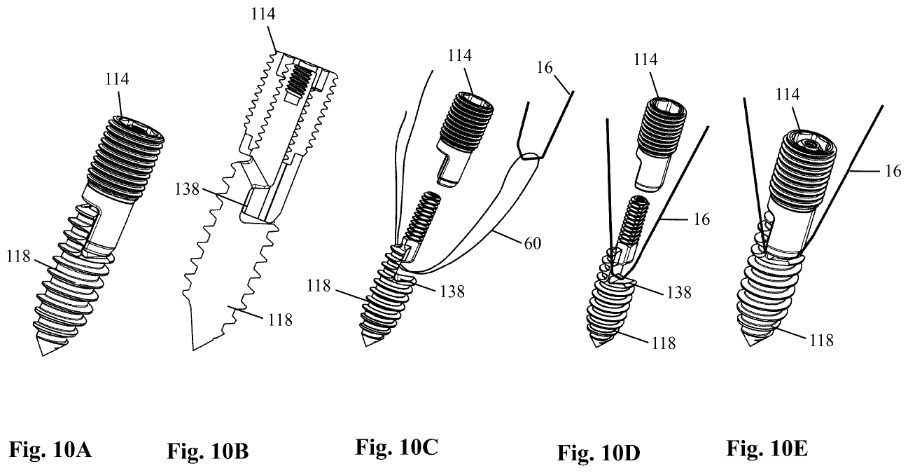

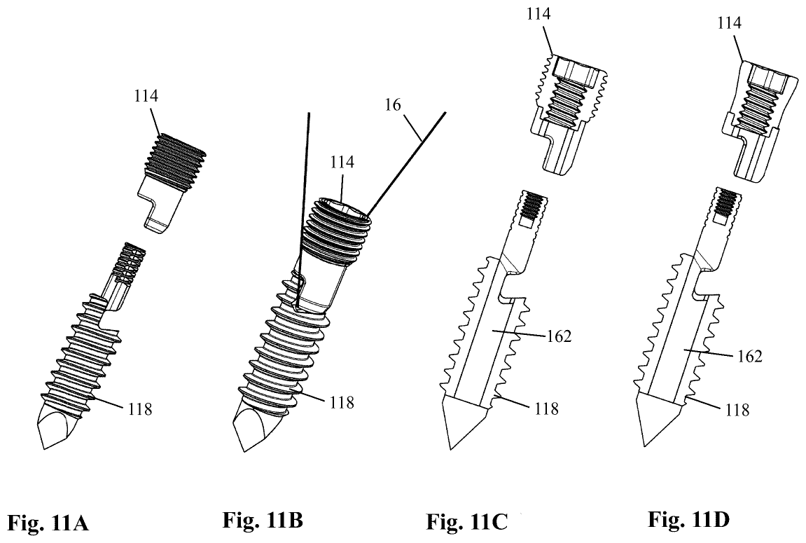

In some embodiments, the distal portion 150 may be manufactured using any of the materials defined to manufacture the knotless anchor assembly 10 or 110, but not limited to the same. In some embodiments, the distal portion 150 may or may not have any threads. In some embodiments, the distal portion 150 may be partial or complete as shown in FIGS. 7A and 10A. By way of example only, he lengths, dimensions may vary depending on the anatomical locations, techniques, tools or other factors that may influence the size of the distal portion 150. In some embodiments, the distal portion 150 may or may not assist with locking the tension members 16. In some embodiments, the distal portion 150 may also create fixation points between itself and the anchor 12. In some embodiments, the distal portion 150 may be partial, as shown in FIGS. 10A-11D.

In some embodiments, the proximal portion 152 may have internal and an external thread that preferably have equal pitches, so that the compression member 114 can thread into bone 5 as it is being threaded onto the post 120. Conversely, in some embodiments, the compression member 114 only may only have internal thread or external thread with variable pitches, thread height, or other varying features. In some embodiments a smooth outside surface allows the tension member 16 to move more easily against it when re-tensioning or adjustments are needed.

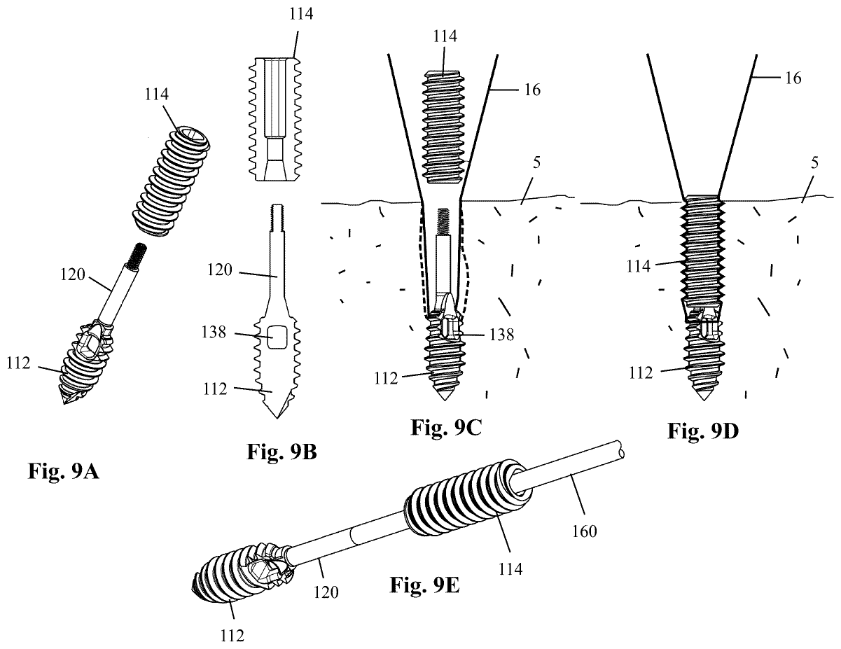

In some embodiments, the passing loop 60 may be inserted into the anchor body 118, and the anchor body 118 driven into bone 5 until completely below the bone surface, as shown in FIGS. 8A-9C. By way of example, the tension member 16 may be inserted into the passing loop 60, which is pulled to pull the tension member 16 through the transverse opening 138, as shown in FIGS. 8A-8D and 9C. Tension is applied to the tension member 16 as needed. To lock the tension member 16 in place, the compression member 114 is inserted and threaded onto the threaded post 120, as shown in FIGS. 8D, 8E, 9C, 9D, both pinching it against the bone 5 and against the anchor body 118. Backing up the compression member 114 unlocks the tension member 16 and adjusts it (i.e. retention) before re-locking. In some embodiments, the tension member 16 may or may not only be locked/pinched between compression member 114 and the anchor 112. In some embodiments, the locking may happen between the distal portion 150 and the anchor body 118 only.

In some embodiments, the compression member 114 may be generally cylindrical and have a size that is complementary to the anchor body recess 128 so that the compression member 114 may be securely mated to the anchor body 118 (e.g., by coupling within the anchor body recess 128). In some embodiments, the distal end 150 has a smooth surface configured to contact the tension member 16 and further configured to create pinch points with the base 132 of the anchor body recess 128 (for example) to secure the tension member 16 to the anchor 112. In some embodiments, the proximal end 152 includes a proximal recess 158 configured to engage with an insertion instrument.

In some embodiments, the compression member 114 is not threaded onto the anchor post 120, but instead the tension member 16 is locked by means of pinching the tension member 16 between the compression member 114 and bone 5 (e.g., the compression member 114 acts as an interference screw), as shown by way of example only in FIG. 9A-9E. In some embodiments, the top of the anchor post 120 may be threaded to facilitate attachment and use of the guide pin 160, as shown in FIG. 9E.

By way of example, the lengths and dimensions of the guide pin 160 may vary depending on the anatomical locations, techniques, tools or other factors that may influence the size of the guide pin 160. In some embodiments, the guide pin 160 may have partial or complete cannulation. In some embodiments, the guide pin 160 may be solid and may have internal or external threads. In some embodiments, the guide pin 160 may connect with the anchor body 118 or anchor post 120 using the threads, as shown in FIG. 9A, press-fit or other mechanisms that are used in the industry to connect tools temporarily or permanently.

In some embodiments, the transverse opening 138 of the anchor body 118 may be open to one side of the anchor body 118, as shown by way of example only in FIGS. 10A-10E. This configuration allows the user to urge the tension member 16 into the transverse opening 138 from the side.

In some embodiments, the anchor body 118 may comprise at least two portions—an anchor body 118 and anchor inner core 162, as shown in FIGS. 11A-11D. In some embodiments, the anchor inner core 162 may be partial or complete.

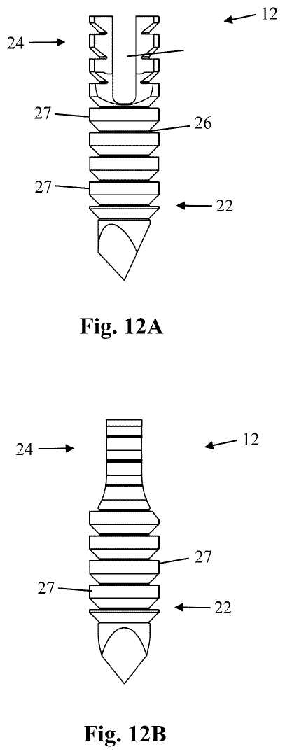

By way of example only, FIGS. 12A and 12B depict an anchor 12 (or anchor 112) that is configured for insertion by tapping the anchor 12 (or anchor 112) in the bone 5 or structure requiring the fixation point. For example, instead of having an outer surface 26 having helical thread enabling a threaded insertion into bone, the anchor 12 shown by way of example in FIGS. 12A and 12B has a plurality of annular directional grooves or ridges 27 that facilitate insertion into bone but resist removal. The compression member 14 and tension member 16, and/or other components described herein may follow the anchor body 18 to complete the locking.

By way of example, FIGS. 12A and 12B represent embodiments of the anchor 12 where the tension member 16 can be tied around any part of the anchor 12, such as, an eyelet, hole, slot, cannulation, or recess, and driven on the bone 5 to complete the repair. The anchor 12 in this embodiment may contain an eyelet or transverse opening in the anchor body 18. If the tension member 16 is not tied it may be connected to the anchor body 18 using other techniques that may be utilized to complete the repair. The anchor 12 may also be inserting using techniques which may capture the tension member 16 outside the anchor body 12 and perform locking. A secondary fixation device may or may not be required to be passed to complete the repair.

The components described herein may be manufactured using standard procedures used in the industry, including, but not limited to, machining, molding, casting, 3D printing, etc.

Recess or eyelets or holes within the anchor 12, knotless anchor assembly 10, pilot tip 20 or any other member that completes the fixation may vary depending upon the anatomical location, number of fixation members required to perform the repair or if the fixation members also include grafts, membrane, gap fillers or other components that may have to be captured at the time of repair or if other devices are used in combination of this knotless anchor assembly 10 to complete the repair.

All embodiments disclosed herein allow repairs using knotless anchor assembly 10 performed in a knotless fashion. A surgeon or other user may tie knots with any or all of the embodiments or partial constructs taught herein.

It is contemplated herein for all embodiments of the knotless anchor assembly that any components may be manufactured out of polymers (including PEEK, PLA, PEKK, UHMWPE and others), metal or metal alloys (including Titanium, Cobalt, Molybdenum, Rhenium, Iron and their alloys), composites (including carbon fiber-, glass fiber-, any fiber-, barium sulfate-and metal-filled polymers), or ceramics (including oxide ceramics, nitride ceramics, diamond, bone, etc.).

In some embodiments of the knotless anchor assembly 10, the insertion sleeve 70 may be connected to or be an integral part of an insertion tool 210, which may have a slot 212 that extends into the body of the insertion tool 210, as shown by way of example only in FIG. 14. In some embodiments, a tension member 16 or passing loop 60 may be located at any point within the open slot 212, which may be used to shuttle tension members 16 and/or other fixation members 214 (e.g. by way of a passing loop 60) into the anchor 12 for fixation. In some embodiments, the entire fixation may be performed using various features built into the insertion tool 212 to accomplish fixation, as depicted by way of example in FIG. 14 or may be performed using a series of tools to perform the fixation. In some embodiments, the anchor body recess 28 of the anchor 12, sleeve slot 74 of the insertion sleeve 70, and slot 212 of the insertion tool 210 may be connected so that a tension member 16 may be introduced to the assembly within the slot 212 of the insertion tool 212 and advanced distally through the sleeve slot 74 and into the anchor body recess 28, wherein it may be secured by a compression member 14 (for example), setscrew, or other locking member.

By way of example only, in a stabilization or other surgical procedure, the anchor 12 of the knotless anchor assembly 10 may be implanted in bone 5. The tension member(s) 16 (e.g. which may comprise one or more loop portions of a single surgical suture, or one or more loop portions from multiple surgical sutures), passing loop 60, and/or fixation members 214 may be shuttled through the insertion tool 210 to the anchor 12 directly. In some embodiments the time of tension member 16, passing loop 60, and/or fixation member 214 shuttling, the insertion tool 210 may be in contact with the anchor 12 through the insertion sleeve 70, shaft or other component.

In some embodiments, the tension member 16 may be driven down through the slot 212 by sliding an insertion tool such as a broach pusher 82 or other pusher device (e.g. introducer or driver 216), driving the tension member 16 down in the anchor 12 to complete the repair or position the tension member 16 for a secondary member 218 or compression member 14 to lock the tension members 16 and complete the repair, as shown in FIG. 14. In some embodiments, the pusher device 216 can be inside or outside the insertion tool 210. In some embodiments, the pusher device 216 may be connected to the insertion sleeve 70. In some embodiments, the mechanism depicted in FIG. 14 may happen outside the anchor 12.

In some embodiments of a method for soft tissue repair requiring an anchor 12 to be implanted into the bone 5 or a tissue requiring fixation, the anchor 12 may provide a fixation point for tensioning or adjustment of the tension member(s) 16, passing loop 60, and/or fixation members 214. In some embodiments, the anchor 12 may include a pilot tip with a transverse hole, eyelet, recess or other feature (e.g. as described above) that can assist with tensioning of the tension members 16.

In some embodiments, as depicted by way of example only in FIGS. 16A and 16B, once adequate tension has been achieved, a secondary anchor body, screw, or compression member 14 may be introduced to perform locking of the tension member 16 and/or fixation members 214. In some embodiments, the anchor 12 may be capable of maintaining purchase within the bone 5 without any assistance or downward pressure from the insertion tool 210 or any other tool used to implant the anchor 12 or retain the anchor 12 within the bone 5 while adjusting the tension in the tension member 16 and/or fixation members 214 and providing strong fixation for introduction of a secondary member 218 and/or another anchor and/or compression member 14 that provides fixation. In some embodiments, the secondary anchor body, screw or compression member 14 component may introduce a secondary member 218 that may be completely disconnected from the anchor 12 in its resting phase prior to fixation. In some embodiments the anchor 12, compression member 14 and/or secondary member 218 may not be connected but can be part of same insertion tool 210 or tool assembly. By way of example, the fixation may either happen at this stage or may require an additional step of passing the secondary member 218 which may be a setscrew, suture, or similar member that may be advanced within the anchor 12.

In some embodiments, a portion of the anchor body 18, for example an additional member 220 such as movable pillars or a wall is configured to translate downward to participate in locking the tension member 16 with the advancement of the compression member 14, as shown by way of example only in FIG. 16B.

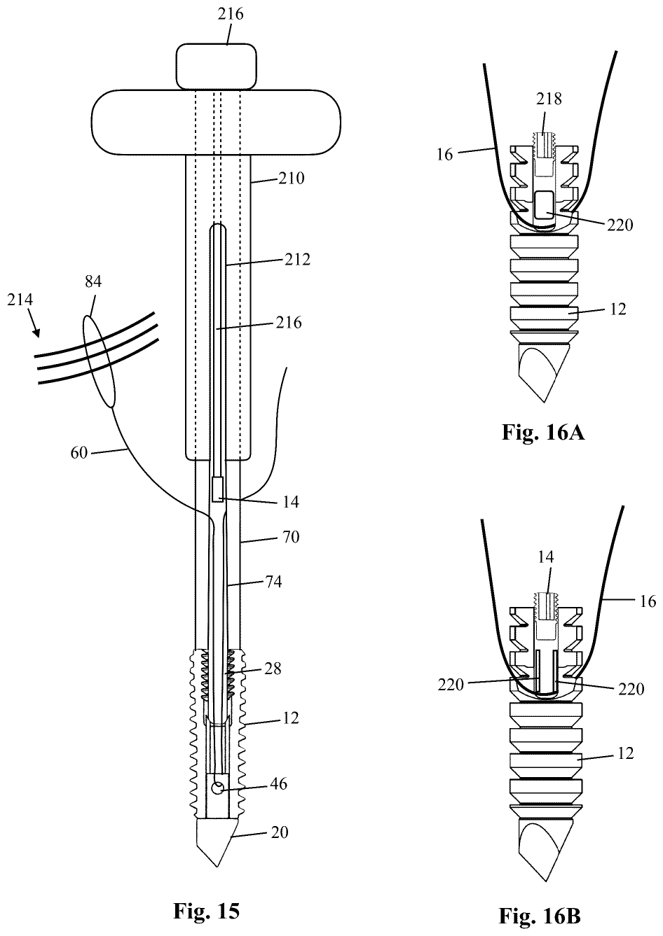

In some embodiments, the anchor 12 may be implanted in the bone 5 using a pilot hole or self-punching mechanism. In some embodiments, a passing loop 60 may be attached to the pilot tip 20 of the anchor 10. In some embodiments, the anchor 12 may be implanted using an internal driver, wherein the internal driver passes into the anchor 12 (e.g. by way of anchor body recess 28). In some embodiments, the anchor 12 may be implanted using an external driver, wherein the external driver is connected to the outer surface of the anchor 12 or the walls of the anchor 12. In some embodiments, the method of fixation involves the tension member 16 or passing loop 60 passing through the transverse hole 46 of the pilot tip 20 or an eyelet or other recess, as shown in FIGS. 5 and 15. In some embodiments, the tension members 16 may exit proximally by way of the slot 212 or other recess in the insertion tool 210 connected to the anchor 12. By way of example only, the slot 212 may be partial, complete or it may be a hole.

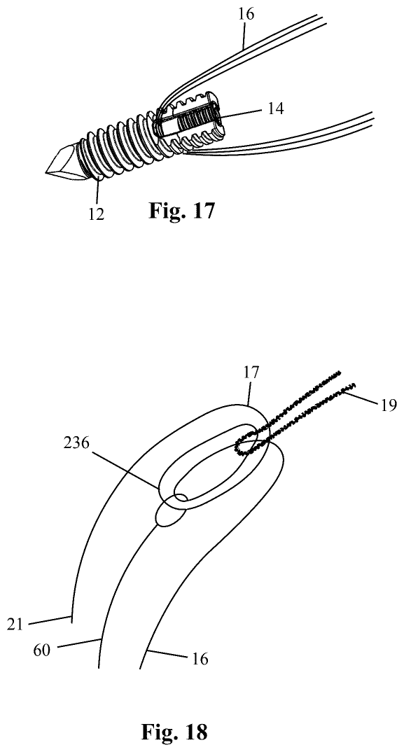

By way of example, fixation members 214 attached to the soft tissues may then be connected to the anchor 12 by passing them through a loop 17 of the tension member 16, looped end 84 of a passing loop 60, or other mechanism configured to connect the tension member 16 or passing loop 60 to the fixation members 214 used for tissue repair. In some embodiments, the fixation members 214 may be pulled into the anchor 12 by the tension member 16 or the passing loop 60. In some embodiments, the insertion tool 210 with a slot 212 or hole along the length, which can be partial or complete and with or without cannulation, may be used to connect the fixation members 214 within the anchor 12. At this point the repair can be completed by using a secondary member 218 or compression member 14 to lock the tension members 16 and/or fixation members 214 within the anchor body, as shown in FIG. 17.

By way of example, for arthroscopic or endoscopic fixation, the tension member 16 and/or fixation sutures 214 may pass through the same cannula or key hole. The insertion shaft or sleeve 70 and/or insertion tool 210 may be made of metal, plastic or other material that can be used to manufacture a shaft 70.

In some embodiments, tension of the fixation members 214 or tension members 16 can be adjusted by pulling on them to the desired tension and then using the compression member 14 to lock them or they can be docked on the same tool or attached to the same tool or a separate tool that may have a tensioner to adjust the tension of the construct, as shown by way of example in FIG. 15.

By way of example, this method requires the tool design and features essential to shuttling of the fixation members 214, as shown in FIG. 15. In some embodiments, the tool design may also assist with tensioning of the fixation members 214. In some embodiments, the compression member 14 may be introduced within the device as a separate step or already connected to the implanted device or tool. In some embodiments, the insertion tool 210 may have a slot 212 that may assist with shuttling of the passing loop 60, and/or tension member 16, and/or fixation members 214. In some embodiments, the passing loop 60 that passes through the insertion tool 210 and pulls the fixation members 214 through that slot 212 may have the ability to maintain tension when its being pulled, as shown in FIG. 15.

By way of example only, this embodiment of the method also requires the use of a shuttling member to connect the tension member 16 to the anchor 12 and requires the insertion tool 210 to assist with connecting the tension member 16 to the anchor 12 or to bring the tension member 16 within the anchor 12 or to a location desired by pulling on the passing loop 60. The tension members 16 may then exit the anchor body recess 28 and the slot 212 of the insertion tool 210 connected to the anchor 12. By way of example, this method may utilize multiple fibers depending on the size of the tissue tear being repaired. In some embodiments, the compression member 14, 114 may be changed in size if desired to fit the anatomy, anchor 12, number of tension members 16, and/or or technique used based on the tissue tear. In some embodiments, the size of the compression member 14 may be changed by a modified modular technique.

In some embodiments, the shuttling or passing loop 60 that may transport the tension members 16 at the site of locking the tension within the fibers may be adjustable. Alternatively, the shuttling or passing loop 60 may not be adjustable.

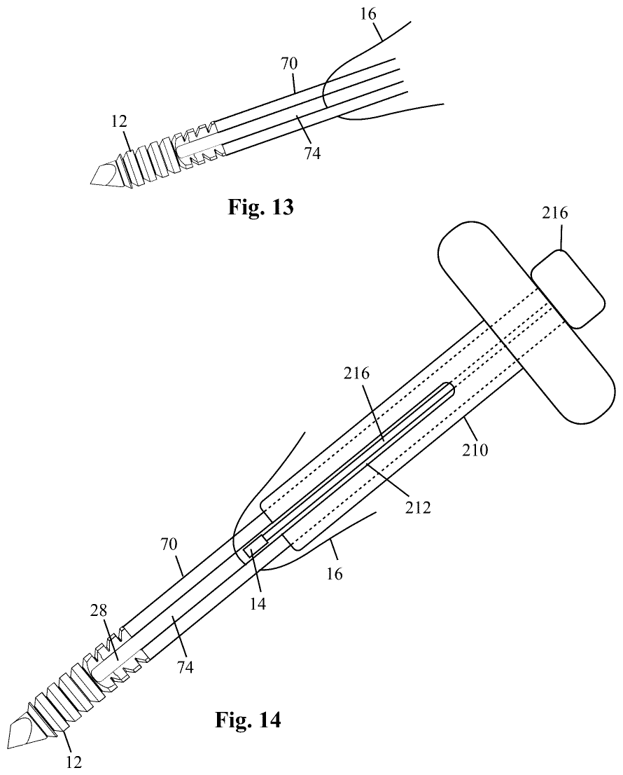

In some embodiments, the knotless anchor assembly 10 taught in FIGS. 13-17 may include an additional member 220 between the anchor 12 and the secondary compression member 218 (or compression member 14). For instance, the additional member 220 may function as a locking member. In some embodiments, the additional member 220 may be deployed within or advances into the anchor 12 after the compression member 14 is driven into the anchor 12. In come embodiments, the additional member 220 traps or locks the tension member 16 and/or fixation members 214 within the anchor 12, as shown in FIG. 16A.

By way of example only, the additional member 220 may vary in shape and can either lock by creating pinching points at the wall of the anchor 12, or base within the anchor 12 or other means of engagement created by advancing the compression member 14 or similar device within the anchor 12. In some embodiments, the compression member 14 or device designed to deploy the additional member 220 may be left in place or removed, as shown in FIG. 16A.

In some embodiments, the additional member 220 may be able to lock the tension members 16 by locking in or becoming a component anchor wall, or the additional member 220 may be a component of the anchor wall that is advanced further by the compression member 14 or similar secondary member 218, as shown by way of example in FIG. 16B.

In some embodiments, the anchor 112 may be inserted by using a push in mechanism or driving the anchor 112 in the bone 5 or tissue that will retain the anchor 112 by using a mechanism to drive or tap it in using a tool instead of implanting it by using a twisting/crewing in motion, as shown in FIG. 8.

An embodiment of this method includes cannulation through the anchor 112, the post 120 connected to the anchor 12, and/or the compression member 114. In some embodiments, the post 120 depicted in FIG. 8 does not have threads for engaging the compression member 114. In some embodiments, the compression member 114 locks or secures fixation members 214 used for soft tissue repair—reconstruction.

In the methods taught herein, the physician or other operator of the knotless suture assembly 10, 110 may actively adjust and re-adjust the fixation of the repair and number of tension members 16 by understanding the size of the tear and number of tension members 16 needed for repair, as shown in FIG. 17. Multiple tension members 16 actively improve the repair after the anchor 12 is seated in the bone 5.

In some embodiments, the fixation members 214 attached to the tissues or used for soft tissue repair may be locked by using a pushing member which is followed by a compression member 14. The pushing member transports the fixation members 214 to the desired location followed by use of the compression member 14, which reaches the resting point within the knotless suture assembly 10, 110. Locking of the construct is completed by the pushing member and compression member 14 performing locking either between itself and the anchor 12 or between itself and the bone 5. The compression member 14 may also perform locking by using a mechanism where it does not have to pass through the anchor 12. For instance, a pulling member or mechanism may be used that accomplishes the same task as a pushing member by locking of the construct or is followed by a compression member 14 to lock the construct.

Tension members 16 and fixation members 218 are interchangeable and only described with different names to assist in the description of use and functionality. Tension members 16 and fixation members 218 are contemplated as being manufactured out of a variety of fibers or filaments including but not limited to polymer filaments (e.g. HMWPE, UHMWPE, PET, PTFE, PEEK, PEKK, PLA, PLLA, etc.), metallic filaments (e.g. Nitinol, Titanium, Titanium alloys, Tantalum, Stainless Steel, etc.) or organic filaments (e.g. Collagen, Silk, etc.) or other filaments such as carbon fiber or carbon nanotubes, etc. Tension members 16 and fixation members 218 are further contemplated to comprise, but not limited to, a coreless suture, a suture with a jacket and a central core, a tape or any other tension member available or contemplated. The length of the tension member 16 is contemplated as being between 150 mm and 1000 mm in length and more preferably between 300 mm and 1000 mm in length and most preferably between 250 mm and 750 mm in length but may chance depending on anatomical location, technique or instrumentation.

In some embodiments, to prevent glove tears, surgeons can use hemostat forceps to roll the tension member 16 during a procedure utilizing the knotless anchor assembly 10.

In some embodiments, the knotless anchor assembly 10 may include a passing member that can change shape and perform locking with or without the assistance of the compression member 14.

In some embodiments, the tension members 16 and a modular button 230 may assist with soft tissue repair or reconstruction with fixation on the surface of bone, tissue or within a tunnel drilled into bone. By way of example only, this method depends on identifying the tear and using the method to repair the soft tissue tear using tension members 16 described herein. By way of example, this technique requires tension members 16 having one or more loops 17. The soft tissue 19 can be wrapped around the loop 17 (e.g., or vice versa) on one end or connected to the loop using mechanisms that can help to secure fixation between the soft tissue and the tension members in the looped orientation 17, as shown in FIG. 18.

It is contemplated herein that the number of loops 17 may vary depending on the size of the tear and strength required to achieve the desired fixation. The determination can me made during the procedure, depending on the type of tear or reconstruction and the number of grafts used, if any.

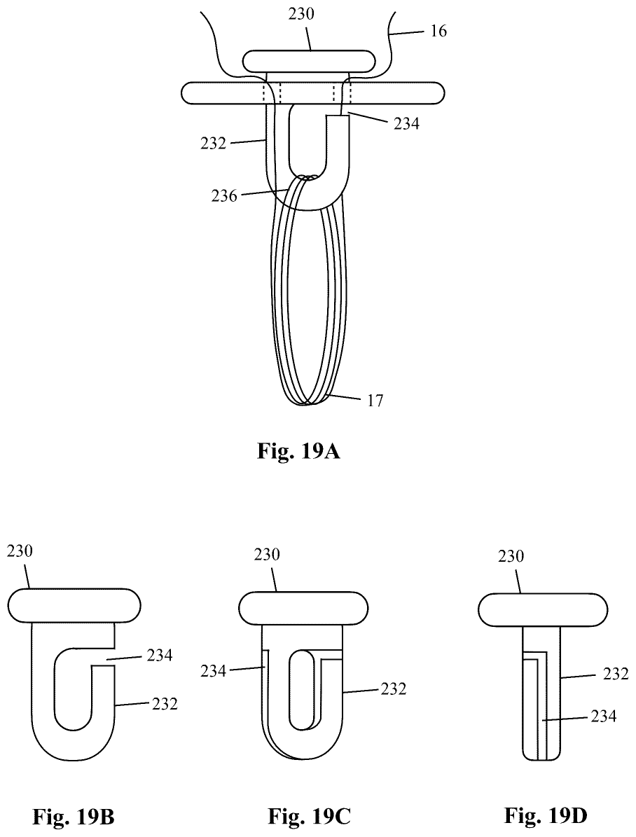

In some embodiments, the opposite loop end may be attached to an anchoring member or device that provides the ability to adjust the length of the tension members 16, adjust the tension within the repair construct, and complete the repair in a knotless fashion. For example, FIG. 19A demonstrates the use of a modular button 230 to achieve the desired outcome.

In some embodiments, the soft tissue is connected to one end of the loop 17 and the other end of the loop is connected to the anchoring member, such as a button 230. In some embodiments, the tension member 16 may also have free ends 21 as part of the construct, which can connect to a button or other fixation device. To be clear, this method does not require the tension members 16 to friction lock in the same manner as a Chinese finger trap and does not require or work by means of locking provided by one tension member 16 woven into another tension member 16.

In some embodiments, the anchor member, such as a button 230, may have a locking pin 232 which has a slot 234 or retaining mechanism to capture the opposite end of the loop 17, as shown in FIG. 19A. By way of example only, the button 230 may be generally similar in form and function to the fixation buttons shown and described in commonly owned and co-pending U.S. Patent Publication No. 2018/0249998, filed 1 Mar. 2018 and entitled “KNOTLESS ORTHOPEDIC STABILIZATION SYSTEM”, the entire contents of which are hereby incorporated by reference into this disclosure as if set forth fully herein.

By way of example only, this embodiment requires pulling tension on the free ends 21 of the tension members 16 to optimize tension in the construct. By way of example only, FIG. 19A depicts a modular button 230 having a locking pin 232 which has a slot 234 and is ‘C’ shaped, as shown by way of example in FIGS. 19B-19D, wherein the looped portions 17 of the tension member 16 may be coupled with the locking pin 232 through the ‘C’ slot 234, as shown in FIG. 18B. In some embodiments, the free ends 21 of the tension members 16 can be passed through the button 230, which has an opening for the free ends 21 to pass. By way of example, tension is applied on the free ends 21 of the tension members 16 to perform locking of the tension members.

This embodiment requires a loop or tension member 16 to perform soft tissue repair using an anchoring member or button with the ability to provide a self-locking, adjustable construct, without any assistance from using a Chinese finger trap or interwoven fibers to provide friction locking. The modular device used on the other end performs the locking of the construct, but it is contemplated that other mechanisms can be used to perform locking away from the soft tissue.

In some embodiments, the button 230 or the anchors 12 used may have cannulation, slots, key holes and can be used from materials used in the industry to manufacture, as long as, adjustable, self-locking can be performed by pulling the free ends 21 of the tension member 16. For example, if an anchor 12 is used instead of a button 230 on the opposite end of the loop 17, the anchor 12 should have the ability to self-lock once tension is applied or by pulling the free ends 21 of the tension members 16. In some embodiments, the free ends 21 of the tension members 16 may be looped and accomplish the same mechanism.

In some embodiments, the free ends 21 of the tension members 16 may or may not be connected to loop ends 17 or passing loop 60 to provide ease and assist with the surgical technique. For example, the looped end 17 on the opposite side of the soft tissue may have a secondary loop 236 on the opposite end of the soft tissue to assist with the surgical technique suture passage or provide additional strength to fixation or other benefits. In some embodiments, the secondary loop 236 may have a tail to assist with passage and the free ends 21 of the tension member 16 may be connected to them if needed.

It is contemplated that all methods and devices taught herein can be performed using open, arthroscopic or minimally invasive techniques with or without using knots in the methods. Is it further contemplated that all components described herein may have a slot, cannulations or key holes.

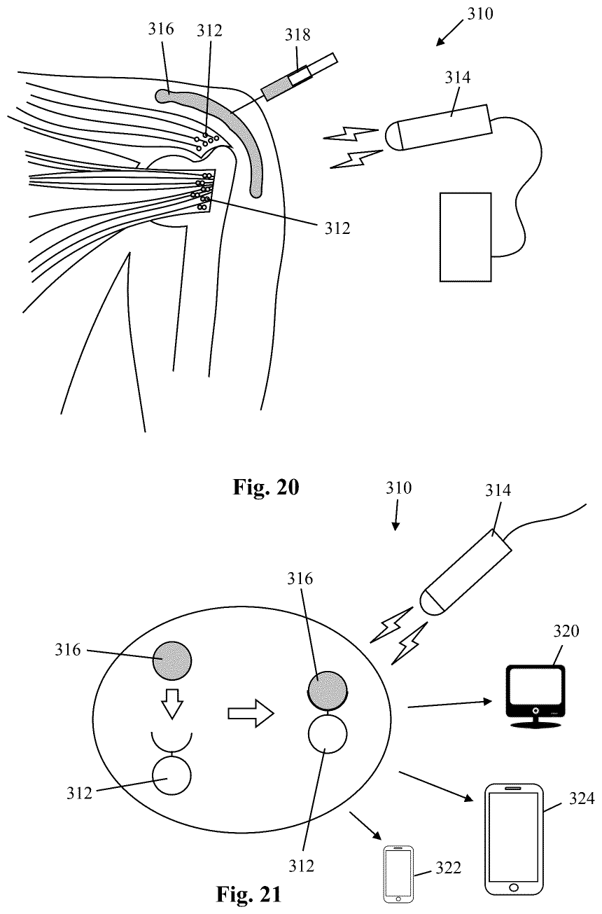

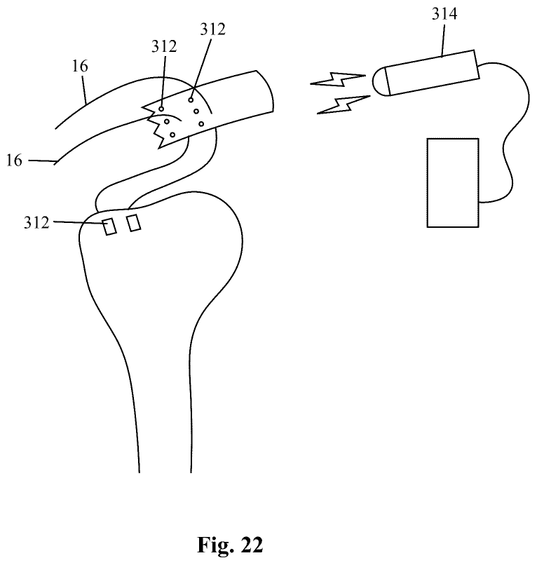

Referring now to FIGS. 20-22, another embodiment of the system includes a system 310 and method for monitoring real-time healing of tissue and other members of the body. By way of example only, the system 310 comprises one or more surgical devices or components, at least one monitoring component, and a detector. The surgical components may be utilized during procedures to aid in the repair of injuries to tissue, tendons, or bone and remain in the body for some period of time after insertion or implantation.

By way of example, during a tissue repair procedure, the surgical component, for example an anchor device, attaches the soft tissue to bone via a secondary surgical component, sutures, to repair an injury. The surgical components work in conjunction with monitoring components which are detected and tracked in real-time to monitor the healing progress of the tissue repair.

In some embodiments, primary surgical components include, but are not limited to, plates, metal, polymer, bio-composite, etc. In some embodiments, secondary surgical components include, but are not limited to, sutures, tapes, textiles, metal, fiber, dye, etc., and these secondary components can be resorbable or non-resorbable.

In some embodiments, the monitoring component(s) 312 functions as a marker of the surgical components securing or affixed to the soft tissue or other member of the body and can include scaffolds, tissue, protein, amino acids, metal, textile, biosensors, or traceable properties of the components themselves, such as traceable sutures. The monitoring components 312 of the primary and secondary surgical components are traceable, visible or detectable in some way, such as, for example, being radio-opaque, emitting biological signals, and/or reacting with naturally present or introduced biologic or chemical agents to produce a detectable reaction. The monitoring components 312 may be also attached to primary or secondary components for repair.

In some embodiments, the signals emitted by the monitoring components 312 may be transmitted and received by a detector or sensor 314, such as, a bio transducer, sonogram, x-ray, or other imaging device to allow instantaneous synthesis and guided treatment planning. The images may be visible on a display device such as a monitor of a computer 320, or computer-implemented portable electronic device such as portable smart phone 322 or tablet computer 324. The signal generated by monitoring components may be converted to a form that is detectable by the sensors.

In some embodiments, the monitoring components 312 can be impregnated with growth factors, steroids antibiotics, and/or pain meds to aid in the healing process, assisting with pain and/or reducing inflammation.

In some embodiments, a computer-implemented electronic mobile device, such as a computer 320, smart phone 322, or tablet computer 324, functions as the detector 314 and obtains and synthesizes the information transmitted by the monitoring components of the surgical components, for example by using one or more of a processor, communications module, and memory storage.

In some embodiments, the monitoring component 312 (e.g., biosensor 312) can be a component of the device used for repair or separately installed on soft tissue. In some embodiments, the biosensor 312 may be selectively permeable to certain components that interact with the biosensor 312 and create a chemical reaction which may later be amplified and detected using different modes of detection. By way of example only, these modes of detection may include, but are not limited to, optical sensors, potentiometric sensors, etc. In some embodiments, the biosensors 312 may be selectively implanted on the soft tissue using either cross linking, adsorption, microencapsulation or other methods used to create a stable position for the sensors. In some embodiments, the biosensors 312 can be stabilized on soft tissue using different methods, such as, cross linking.

In some embodiments, the biosensors 312 may interact with the desired components and create a chemical reaction which can help identify the location, assist with detection of sensor, and/or release growth factors or other agents, which can assist with healing of the soft tissue or agents and reduce the inflammation or perform desired functions.

In an embodiment of the method of repair disclosed herein, the biosensor 312 is either a component of the device or implanted on soft tissue at the time of repair. In some embodiments, the detection of re-tear or integrity of the repair can be performed at a later stage by injecting components which have the ability to interact with the biosensors and create an electro-chemical reaction, which can be detected using an optical sensing device or similar technology, as depicted by way of example only in FIG. 20.

In some embodiments, the implanted biosensors 312 can a made out of carbon nanotubes or similar materials or other materials that are used to develop biosensors which have the ability to create an electro-chemical reaction which can be detected, e.g. infrared signals. etc. By way of example only, the biosensors 312 can be antibodies, enzymes, cells, DNA or any other synthetically designed material to improve stability and improve detection.

By way of example, this system 310 not only functions to monitor and track healing but also detect future injuries and repair failures since the location of the components is monitored and detected. For instance, a displaced anchor or torn tissue would be instantly noticed, which could change the extent of the injury and direction of the treatment plan. Thus, this system 310 reduces delays in treatment and may offer assessment during dynamic testing.

In an exemplary embodiment of the invention, a physician may use a method other than open and scope or MRI to diagnose the tear. To perform the repair in this instance, the physician may inject the components or agents 316 that combine with the receptors or biosensors 312 on the tissue that were either placed of the time of repair or is naturally occurring. As shown in FIG. 20, the biosensor 312 is bound to the agent 316 that has been injected into the tissue by injector 318. This combination causes an electrochemical reaction that can be detected by different means, including, but not limited to, optical sensor, electrochemical sensor, or other method, to diagnose the location and activity of the combined sensor 312 and agent 316 and tissue the combined sensor and agent are attached to, as shown by way of example in FIG. 21. This diagnosis will help the surgeon actively change treatment plans, for example, determining whether the patient requires surgery or should go to physical therapy, etc.

It is contemplated herein for all embodiments of the primary surgical components that any components may be manufactured out of polymers (including PEEK, PLA, PEKK, UHMWPE and others), metal or metal alloys (including Titanium, Cobalt, Molybdenum, Rhenium, Iron and their alloys), composites (including carbon fiber-, glass fiber-, any fiber-, barium sulfate-and metal-filled polymers), or ceramics (including oxide ceramics, nitride ceramics, diamond, bone, etc.) or other materials used in the industry.

The secondary surgical components can be any member capable of passing through the anchor or connecting to the anchor or applicable component, including, but not limited to, fiber, suture, tape, tissue graft, polymer, metal or wires or other materials used in the industry.

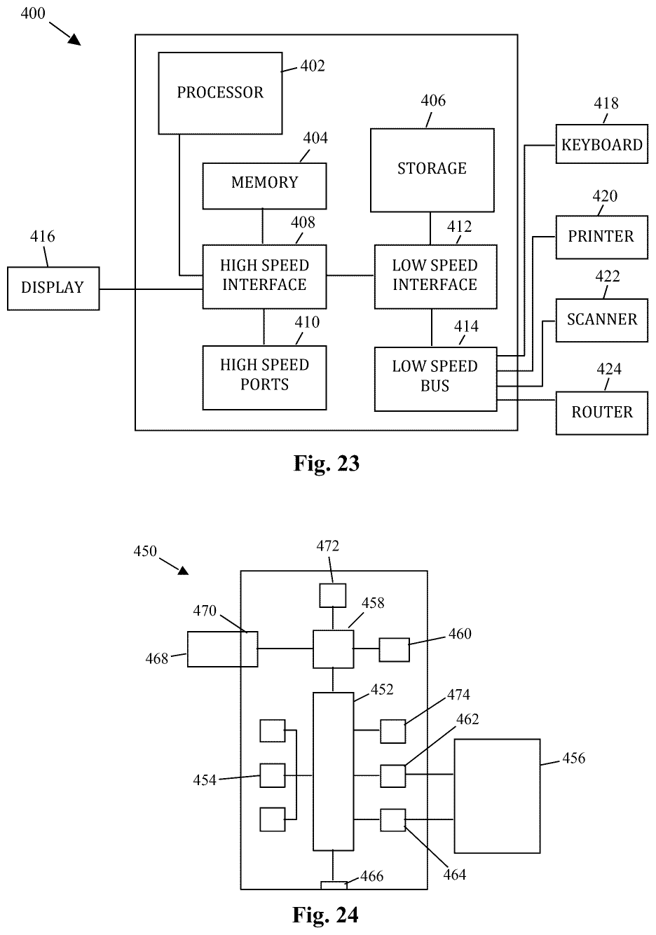

FIGS. 23-24 are example block diagrams of computer-implemented electronic devices 400, 450 that may be used to implement the systems and methods described in this document, as either a client or as a server or plurality of servers. Computing device 400 is intended to represent various forms of digital computers, such as laptops, desktops, workstations, personal digital assistants, servers, blade servers, mainframes, and other appropriate computers. Computing device 450 is intended to represent various forms of mobile devices, such as personal digital assistants, cellular telephones, smart-phones, and other similar computing devices. In this example, computing device 450 may represent a portable or hand-held computing device such as a smart phone 322 or tablet computer 324, while computing device 400 may represent stationary computer 320 referenced in this disclosure. The components shown here, their connections and relationships, and their functions, are meant to be examples only, and are not meant to limit implementations described and/or claimed in this document.

Referring to FIG. 23, computing device 400 includes a processor 402, memory 404, a storage device 406, a high-speed interface 408 connecting to memory 404 and high-speed expansion ports 410, and a low speed interface 412 connecting to low speed bus 414 and storage device 406. Each of the components 402, 404, 406, 408, 410, and 412, are interconnected using various busses, and may be mounted on a common motherboard or in other manners as appropriate. The processor 402 can process instructions for execution within the computing device 400, including instructions stored in the memory 404 or on the storage device 406 to display graphical information for a graphic user interface (GUI) on an external input/output device, such as display 416 coupled to high-speed interface 408. In other implementations, multiple processors and/or multiple buses may be used, as appropriate, along with multiple memories and types of memory. Also, multiple computing devices 400 may be connected, with each device providing portions of the necessary operations (e.g., as a server bank, a group of blade servers, or a multi-processor system).

The memory 404 stores information within the computing device 400. By way of example only, the memory 404 may be a volatile memory unit, non-volatile memory unit, or another form of computer-readable medium, such as a magnetic or optical disk (for example).

The storage device 406 is capable of providing mass storage for the computing device 400. In one implementation, the storage device 406 may be or contain a non-transitory computer-readable medium (e.g., any and all computer-readable media except transitory, propagating signals), such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer-or machine-readable medium, such as the memory 404, the storage device 406, or memory on processor 402.

The high-speed interface 408 manages bandwidth-intensive operations for the computing device 400, while the low speed interface 412 manages lower bandwidth-intensive operations. Such allocation of functions is by way of example only. In one implementation, the high-speed interface 408 is coupled to memory 404, display 416 (e.g., through a graphics processor or accelerator), and to high-speed expansion ports 410, which may accept various expansion cards (not shown). In the implementation, low-speed interface 412 is coupled to storage device 406 and low-speed expansion port 414. The low-speed expansion port may include various communication ports (e.g., USB, Bluetooth, Ethernet, wireless Ethernet) and may be coupled to one or more input/output devices, such as a keyboard 418, a printer 420, a scanner 422, or a networking device such as a switch or router 424, e.g., through a network adapter.

The computing device 400 may be implemented in a number of different forms. For example, it may be implemented as a standard server, or multiple times in a group of such servers. It may also be implemented as part of a rack server system. In addition, it may be implemented in a personal computer such as a laptop computer. Alternatively, components from computing device 400 may be combined with other components in a mobile device, such as device 450 (FIG. 24). Each of such devices may contain one or more of computing device 400, 450, and an entire system may be made up of multiple computing devices 400, 450 communicating with each other.

Referring to FIG. 24, computing device 450 includes a processor 452, memory 454, an input/output device such as a display 456, a communication interface 458, and a transceiver 460, among other components. The device 450 may also be provided with a storage device, such as a microdrive or other device, to provide additional storage. Each of the components 450, 452, 454, 456, 458, and 460, are interconnected using various buses, and several of the components may be mounted on a common motherboard or in other manners as appropriate.

The processor 452 can execute instructions within the computing device 450, including instructions stored in the memory 454. The processor may be implemented as a chipset of chips that include separate and multiple analog and digital processors. Additionally, the processor may be implemented using any of a number of architectures. For example, the processor 452 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 450, such as control of user interfaces, applications run by device 450, and wireless communication by device 450.

The processor 452 may communicate with a user through control interface 462 and display interface 464 coupled to a display 456. The display 456 may be, for example, a TFT (Thin-Film-Transistor Liquid Crystal Display) display or an OLED (Organic Light Emitting Diode) display, or other appropriate display technology. The display interface 464 may comprise appropriate circuitry for driving the display 456 to present graphical and other information to a user. The control interface 462 may receive commands from a user and convert them for submission to the processor 452. In addition, an external interface 466 may be provided in communication with processor 452, so as to enable near area communication of device 450 with other devices. External interface 466 may provide, for example, for wired communication in some implementations, or for wireless communication in other implementations, and multiple interfaces may also be used.

The memory 454 stores information within the computing device 450. The memory 454 can be implemented as one or more of a computer-readable medium or media, a volatile memory unit or units, or a non-volatile memory unit or units. Expansion memory 468 may also be provided and connected to device 450 through expansion interface 470, which may include, for example, a SIMM (Single In Line Memory Module) card interface. Such expansion memory 468 may provide extra storage space for device 450, or may also store applications or other information for device 450. Specifically, expansion memory 468 may include instructions to carry out or supplement the processes described above, and may include secure information also. Thus, for example, expansion memory 468 may be provided as a security module for device 450, and may be programmed with instructions that permit secure use of device 450. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, cause performance of one or more methods, such as those described above. The information carrier is a computer-or machine-readable medium, such as the memory 454, expansion memory 468, or memory on processor 452 that may be received, for example, over transceiver 460 or external interface 466.

Device 450 may communicate wirelessly through communication interface 458, which may include digital signal processing circuitry where necessary. Communication interface 458 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 460. In addition, short-range communication may occur, such as using a Bluetooth, WiFi, or other such transceiver (not shown). In addition, GPS (Global Positioning System) receiver module 472 may provide additional navigation-and location-related wireless data to device 450, which may be used as appropriate by applications running on device 450.

Device 450 may also communicate audibly using audio codec 474, which may receive spoken information from a user and convert it to usable digital information. Audio codec 474 may likewise generate audible sound for a user, such as through a speaker, e.g., in a handset of device 450. Such sound may include sound from voice telephone calls, may include recorded sound (e.g., voice messages, music files, etc.) and may also include sound generated by applications operating on device 450.

The computing device 450 may be implemented in a number of different forms, some of which are shown in the figure. For example, it may be implemented as a cellular telephone. It may also be implemented as part of a smart-phone, personal digital assistant, or other similar mobile device.

Additionally computing device 400 or 450 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all sub-ranges and individual values are intended to be encompassed. This invention is not to be limited by the embodiments disclosed, including any shown in the drawings or exemplified in the specification, which are given by way of example and not of limitation. Additionally, it should be understood that the various embodiments of the knotless anchor assembly described herein contain optional features that can be individually or together applied to any other embodiment shown or contemplated here to be mixed and matched with the features of that device.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

What is claimed is:

1. A system for monitoring healing of tissue or other members of a patient's body, comprising:
a surgical component utilized in a surgical procedure to aid in the repair of injuries to tissue, tendons, or bone, the surgical component configured to remain in the body for a period of time after completion of the surgical procedure;
a monitoring component associated with the surgical component and having a traceable property that, when activated, is detectable to indicate a location of the monitoring component; and
a detector configured to detect the activated traceable property of the activated monitoring component.

2. The system of claim 1, wherein the surgical component comprises an anchor device.

3. The system of claim 2, wherein the anchor device comprises at least one of a plate, metal polymer, bio-composite, suture, tape, textile, metal, fiber, and dye.

4. The system of claim 1, wherein the monitoring component comprises at least one of a biosensor, scaffold, tissue, protein, amino acid, metal, and textile.

5. The system of claim 4, wherein the traceable property comprises a biochemical reaction to one or more biochemical agents present in the proximity of the monitoring component.

6. The system of claim 5, wherein the biochemical agent is at least one of:
injected by a user near the monitoring component to activate the traceable property; and
produced by the patient's body during a healing process.

7. The system of claim 1, wherein the traceable feature is an optically traceable feature of the monitoring component.

8. The system of claim 1, wherein the monitoring component is one of attached to and integral with the surgical component.

9. The system of claim 1, wherein the monitoring component is located on tissue near the surgical component.

10. The system of claim 1, wherein the monitoring component contains a medicament configured to at least one of aid in the healing process, assist with pain management, and reduce inflammation near the surgical component.

11. The system of claim 10, wherein the medicament is at least one of growth factors, steroids, antibiotics, and pain medication.

12. The system of claim 10, wherein the monitoring component is configured to release the medicament in response to an activation signal received by the monitoring component.

13. The system of claim 12, wherein the activation signal comprises a signal initiated by a user having an activation device.

14. The system of claim 13, wherein the activation signal comprises a biochemical reaction with a biochemical agent present in the vicinity of the monitoring component.

15. The system of claim 1, further comprising an electronic mobile device in communication with the detector, the electronic mobile device including a processor and a non-transient computer-readable storage media containing a set of instructions that, when executed by the processor, cause the electronic mobile device to:
receive data from the detector pertaining to the activated traceable component of the monitoring component;
analyze the data pertaining to the activated traceable component of the monitoring component to determine the location of the activated monitoring component; and
display, on a display device, the relative location of the activated monitoring component and the surgical component.

16. The system of claim 15, wherein the set of instructions, when executed by the processor, further cause the electronic mobile device to:
determine the occurrence of at least one of a healing event and a re-tear event.

17. The system of claim 15, wherein the detector forms a part of the electronic communication device.

18. The system of claim 15, wherein the display device forms a part of the electronic communication device.

19. The system of claim 15, wherein the electronic communication device comprises at least one of a smart phone, computer, laptop computer, and a tablet computer.

20. A method of monitoring healing of tissue or other members in a patient's body, comprising:
providing a system for monitoring healing of tissue or other members in the patient's body, the system comprising: (i) a surgical component configured for use in a surgical procedure to aid in the repair of injuries to tissue, tendons, or bone, the surgical component configured to remain in the body for a period of time after completion of the surgical procedure; (ii) a monitoring component associated with the surgical component and having a traceable property that, when activated, is detectable to indicate a location of the monitoring component; and (iii) a detector configured to detect the activated traceable property of the activated monitoring component;
implanting the surgical component into the patient's body during a surgical procedure to repair an injury to at least one of tissue, tendons, and bone;
implanting at least one of the monitoring components within tissue, tendons, or bone in the vicinity of the surgical component;
detecting the activated monitoring component; and
determining the location of the activated monitoring component; and
determining the occurrence of at least one of a healing event and a re-injury event based upon the determined location of the activated monitoring component.
